(19) United States
(12) Patent Application Publication
(10) Pub. No.: US 2023/0028983 A1
(43) Pub. Date: Jan. 26, 2023

(54) PROTEIN FAMILIES MAP

(71) Applicant: BenevolentAI Technology Limited, London (GB)

(72) Inventor: Oliver Oechsle, London (GB)

(73) Assignee: BenevolentAI Technology Limited, London (GB)

(21) Appl. No.: 17/786,939

(22) PCT Filed: Dec. 9, 2020
(86) PCT No.: PCT/GB2020/053155

§ 371 Date: Jun. 17, 2022

(30) Foreign Application Priority Data
Dec. 20, 2019 (GB) 1919102.2

(51) Int. Cl. G16B 45/00; G06F 16/901; G06F 16/904; G06F 16/9038; G16B 20/00

(52) U.S. Cl. CPC G16B 45/00; G06F 16/904; G06F 16/9024; G06F 16/9038; G16B 20/00

(57) ABSTRACT

Methods, apparatus, system and computer-implemented method are provided for a computer-implemented method of identifying candidate entities of interest associated with disease selection information. The method including: receiving a first set of entities that are predicted to be associated with the disease selection information; retrieving a second set of entities that are known to be associated with the disease selection information; generating a set of entity mappings between entities of the first set of entities, entities the second set of entities, and entities of a graph structure in relation to the disease selection information, the graph structure based on an entity hierarchy, ontology or taxonomy of an entity family associated with the first and second sets of entities, linking entities from the first and second sets of entities to the graph structure based on the generated set of entity mappings; and identifying candidate entities of interest from those linked entities of the first and second sets of entities on the graph structure based on determining where each entity from the first set of entities is located on the graph structure relative to one or more entities of the second set of entities on the graph structure.

The present application relates to apparatus, system(s) and method(s) for identifying candidate entities of interest associated with disease selection information or entity selection information.

### BACKGROUND

During drug discovery, scientists are tasked with identifying potential drugs that could be used to treat a disease of interest (and their underlying relationship). More often than not, scientists become encumbered by having to review and research too many or large numbers of targets, genes, drugs and/or compounds before arriving at a selected few that may be potential drugs. Typically, databases are queried in relation to, without limitation, for example the disease of interest; the query may return a result set including a set of entities or pairs of entities that have an association with the disease of interest. The result set is typically displayed or provided to the user in a standard list-based result format or a grid-based result format, which requires the user and/or system to further curate and filter unwanted entities with unwanted properties (e.g. controlled substances, illegal drugs, oral drugs, intravenous etc.).

For example, scientists interested in a particular disease may want to generate a list of molecules/drugs that are potentially novel for treatment of that disease. They may query a knowledge database in relation to disease that returns a result set of molecules/drugs or any other result set comprising a plurality of entities associated with the disease (e.g. disease affects gene/protein X; disease treated by drug D1), and/or a plurality of entity pairs (e.g. drug D1 is associated with target/gene/protein X) associated with the disease or any other type of association.

Conventionally the result set may be displayed as a standard tabulated results page with different filters for removing entities with various unwanted properties the scientist is not interested in (e.g. controlled substance, illegal drug, oral drugs, intravenous etc.). Essentially the search result list contains a plurality of entities associated with the disease. Although a user may inspect the search result list, such as by way of example only but not limited to, inspect possible molecules of interest (e.g. 150-1000 molecules), they have to triage them in an attempt to determine candidate entities of interest that may not have been used to classically treat the disease. This can be a time consuming process. Given the large number of possible results in the search result list and the requirement, there are a lot of potential errors that can creep into the process whereby an important hypothesis, or a poor choice of candidate molecules/drugs, or entities may be made resulting in costly and wasted time and resources in pursuing further research and development of a poor candidate set of entities.

Essentially, triaging the queried results to determine particular entities of interest (unidentified prior) poses difficulties in practice due to the volume of results of the result set. Machine learning (ML) methods such as a Bayesian network may be useful in addressing some of these difficulties associated. However, these methods tend to be slow and not without deficiencies. For instance, the present ML methods lack suitable mappings that would permit a robust search through the underlying data - overlooking important hypotheses or delivering a poor selection of entities. In effect, it may not just be costly, but indeed wasteful of time and resource that could be devoted to further research and development.

There is a desire for a mechanism, process or apparatus capable of efficiently and concisely searching for entities associated with disease selection information and generating an efficient and concise representation for assisting and/or use in identifying candidate entities of interest for development during, without limitation, for example drug discovery, optimisation and the like and/or as the application demands.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides methods, apparatus and/or systems for processing search results based on predicted entities associated with entity selection information or a search query and known entities associated with the entity selection information or search query in relation to an entity family is associated with the predicted or known entities. The search results are processed to determine those predicted and known entities that are associated, related and/or map onto entities of the entity family. The processed search results are output as a graph structure, where the graph structure is based on the entity family associated with a hierarchy, taxonomy, or ontology, which is capable of being represented as a linked graph structure. One or more of the predicted entities and/or one or more of the known entities that are mapped onto entities of the entity family are linked to the corresponding entity nodes of the graph structure of the entity family. The linked predicted and known entities are overlaid on the graph structure for use in identification of candidate entities of interest.

In a first aspect, the present disclosure provides a computer-implemented method of identifying candidate entities of interest associated with disease selection information or entity selection information, the method including: receiving a first set of entities that are predicted to be associated with the disease selection information or entity selection information; retrieving a second set of entities that are known to be associated with the disease selection information or entity selection information; generating a set of entity mappings between entities of the first set of entities, entities the second set of entities, and entities of a graph structure in relation to the disease selection information or entity selection information, the graph structure based on an entity hierarchy, ontology or taxonomy of an entity family associated with the first and second sets of entities; linking entities from the first and second sets of entities to the graph structure based on the generated set of entity mappings; and identifying candidate entities of interest from those linked entities of the first and second sets of entities on the graph structure based on determining where each entity from the first set of entities is located on the graph structure relative to one or more entities of the second set of entities on the graph structure.

As an option, the method further including: overlaying the linked entities on the graph structure. As another option, overlaying may include displaying the graph structure with an overlay associated with the linked entities. As an option, the step of overlaying is performed prior to identifying the candidate entities of interest. Additionally or alternatively, as an option, the step of overlaying is performed after identifying the candidate entities of interest.

Optionally, determining sets of entity mappings is based on determining entity associations between entities of the first set of entities, entities of the second set of entities, and/or family entities of the graph structure.

As an option, the set of entity mappings further comprising any one or more entity mappings from the group of: an entity mapping between an entities of the first set of entities, wherein the entities of the entity mapping are determined to be associated by an entity relationship therewith; an entity mapping between an entities of the second set of entities, wherein the entities of the entity mapping are determined to be associated by an entity relationship therewith; an entity mapping between an entity of the first set of entities and an entity of the second set of entities, wherein the entities of the entity mapping are determined to be associated by an entity relationship therewith; an entity mapping between an entity of the first set of entities and a family entity of the graph structure, wherein the entities of the entity mapping are determined to be associated by an entity relationship therewith; and an entity mapping between an entity of the second set of entities and a family entity of the graph structure, wherein the entities of the entity mapping are determined to be associated by an entity relationship therewith.

Optionally, identifying candidate entities of interest further comprises identifying candidate entities of interest in the graph structure based on a positioning between those entities of the first set of entities added to the graph structure and those entities of the second sets of entities added to the graph structure.

As an option, adding each entity from the first and second sets of entities as entity nodes to the graph structure is based on predicted entity associations between the entity and a family entity in the graph structure corresponding to the ontological, hierarchical and/or taxonomic entity family.

Optionally, adding each entity from the first and second sets of entities as entity nodes to the graph structure is based on known associations between said each entity and an family entity in the graph structure corresponding to the ontological, hierarchical and/or taxonomic entity family.

As another option, graphically weighting each entity node linked to the graph structure based on a confidence score associated with each entity from the first and/or second set of entities, wherein the graphically weighting may include weighting the size, colour, shape, and other metadata associated with the entity nodes based on the corresponding confidence score. As an option, the confidence score is indicative of the prediction strength associated with the entity nodes.

Optionally, adding an entity from the first and second sets of entities to the graph structure further comprises adding said entity to the graph structure as an entity node when an indirect entity association exists between said entity and at least one other entity of the first and second sets of entities having an association with an family entity of the graph structure.

As an option, the method further comprising: identifying an indirect association between said entity and said entity of the graph structure based on a determined entity association between said entity and another entity of the first or second set of entities having a direct or indirect association with said family entity of the graph structure; and linking the entity by adding it to said another entity of the first or second set of entities.

Optionally, the disease selection information includes data representative of at least one from the group of: one or more diseases, one or more symptoms of the one or more diseases, one or more cell types associated with the one or more diseases, one or more tissue types associated with the one or more diseases, one or more organs associated with the one or more disease, one or more biological parts associated with the one or more diseases, or one or more disease processes associated with the one or more diseases.

As an option, an entity comprises entity data associated with an entity type from at least the group of: gene; disease or disease process(es); compound/drug; protein; chemical, organ, biological part, tissue, cell, treatments and/or other therapies; and/or any other entity type associated with bioinformatics, chem(o)informatics, biology, biochemistry, chemistry, medicine, pharmacology, and/or any other field relevant to diagnostic, and/or drug discovery and the like.

As another option, the first set of entities are associated with an entity type from the group of: diseases, disease process(es) and the like; the second set of entities are associated with an entity type from the group of: drugs, chemicals, compounds, pharmacology, treatments and/or other therapies and the like; and the family entities of the graph structure are associated with an entity type from the group of: proteins, genes, diseases and/or disease processes.

As an option, receiving the first set of entities further comprises receiving a predicted set of entities output from one or more machine learning, ML, model(s) or entity identification system(s) configured for predicting or identifying entities associated with the disease selection information; receiving the second set of entities further comprises receiving the second set of entities from retrieving entities known to be associated with the disease selection information from one or more content sources, a content source including data representative of entities known to be associated with disease selection information; generating the graph structure further comprises generating the graph structure based on an entity family or family type associated with an ontology, hierarchy and/or taxonomy of family entities, wherein the graph structure comprises a plurality of entity family nodes, each entity family node representing a family entity associated with the entity family; adding entities to the graph structure further comprising populating the graph structure based on one or more entity mappings between the predicted entities of the first set of entities, between the known entities of the second set of entities, between the predicted entities and known entities of the first and second sets of entities, and/or between the predicted or known entities of the first or second sets and the family entities of the graph structure, and linking one or more entities of those entity mappings associated with an entity family node to the graph structure; and identifying candidate entities of interest from the populated graph structure based on the positioning between the predicted entities and the known entities added to the graph structure.

As an option, determining entity mappings further comprises determining interacting entity pairs from the first set of entities, the second set of entities, entity family of the graph structure, and/or disease selection information, wherein an entity pair comprises at least a first entity and a second entity and an entity relationship associated therewith.

Optionally, each interacting entity pair comprises one or more from the group of: an entity of a first type and an entity of a second type having a predicted relationship therewith, wherein the entity of the second type corresponds to an entity type associated with the family entity of the graph structure; an entity of a first type and an entity of a second type having a known relationship therewith, wherein the entity of the second type corresponds to an entity type associated with the family entity of the graph structure.

As an option, the method further comprising displaying the linked hierarchical tree structure with indications of predicted entities being linked directly or indirectly with one or more nodes corresponding to family entities of the graph structure for identifying candidate entities of interest.

As an option, the step of retrieving the second set of entities further comprising retrieving a set of known interacting entity pairs from the content source.

Optionally, each entity pair of the set of known interacting entity pairs comprises a first entity of a first entity type and a second entity corresponding to an entity type associated with an ontological hierarchy or taxonomy of the second set of entities different to the first entity type.

Optionally, the first type of entities correspond to entities associated with drugs, compounds, chem(o)informatics, genes of other drug targets, and/or other therapies; the second type of entities correspond to entities associated with protein and/or gene families; and the candidate entities of interest correspond to entities of the first type of entities.

As an option, the relationship between each entity pair of the set of predicted interacting biological entities comprises a relationship based on the first entity of the first entity type and the second entity of the second entity type in relation to a disease associated with the disease selection information.

As another option, the method further including receiving a user input associated with selecting at least one of: a disease, symptoms of a disease and/or a disease process; and generating disease selection information based on the user input.

As an option, the one or more ML model(s) comprises a relational ML model configured for predicting pairs of interacting entities associated with the disease selection information. As an option, the relational ML model is trained based on a machine learning technique using labelled training datasets and/or rulesets for predicting pairs of interacting entities associated with disease selection information.

As an option, the one or more ML model(s) are configured for predicting pairs of interacting entity pairs associated with disease selection information from a corpus of text, the corpus of text comprising a large scale document repository including a plurality of documents, articles, literature, web-sites and/or any other digital information and/or data associated with disease selection information, entities of the first type, entities of the second type and/or entities of the second set of entities.

As an option, further comprising detecting clusters of candidate entities of interest based on performing automatic cluster recognition in relation to entities of the first set of entities and the entities of the second set of entities added to the graph structure; and indicating the clusters of candidate entities of interest for identifying possible candidates of interest.

As an option, indicating the clusters of candidate entities of interest further comprises displaying the clusters of candidate entities of interest as an overlay over the graph structure.

Optionally, the method further comprising performing automatic cluster recognition based on inputting the linked graph structure into an automatic cluster recognition ML model configured for predicting locations or regions of interest in the linked graph structure associated with candidates of interest; and displaying indications of the predicted locations or regions of interest as an overlay over the hierarchical tree structure for identifying candidate entities of interest.

Optionally, generating an automatic cluster recognition ML model based on training an ML technique to locate regions of interest in the linked graph structure associated with candidates of interest using a labelled training dataset, wherein the labelled training dataset for training the model based on an ML technique is based on one or more user interactions of selecting candidate entities of interest from the multiple linked graph structures derived from multiple search queries in relation to multiple disease selection information.

As an option, generating an automatic cluster recognition ML model based on training an ML technique to locate regions of interest in the linked graph structure associated with candidates of interest using a rule set associated with locating regions of interest in the linked graph structure in relation to candidate entities of interest.

As an option, the method further comprising updating the automatic cluster recognition ML model based on user interactions with the generated and linked graph structure.

Optionally, the method further comprising performing automatic cluster recognition based on data representative of a rule set comprising at least one or more rules from the group of: a rule associated with defining a region of interest corresponding to region of the graph structure in which an entity of the first type is linked to an entity of the second type being distant to other entities of the first type; a rule associated with defining a region of interest corresponding to a region of the graph structure in which an entity of the first type being linked to an entity of the second type, both of which have a predicted association with each other or the graph structure; a rule associated with defining a region of interest corresponding to a region of the graph structure in which the association between an entity of the first type being linked to an entity of the second type is predicted; a rule associated with defining a region of interest corresponding to a region of the graph structure in which the disease is sufficiently distant from the known treatment(s) of an existing drug; and a rule associated with defining a region of interest corresponding to a region of the graph structure in which a gene that a drug claims to be targeting is also not known to be associated with that disease.

In a second aspect, the present disclosure provides an apparatus comprising a processor unit, a memory unit and a communication interface, the processor unit connected to the memory unit and the communication unit, wherein the apparatus is configured to implement the computer-implemented method according to any of the features of the first aspect, combinations thereof, modifications thereto, and/or as herein described.

In a third aspect, the present disclosure provides a system comprising: a user interface configured for receiving data representative of disease selection information; a candidate entity identification apparatus according to any of the features of the second aspect, combinations thereof, modifications thereto, and/or as herein described, the candidate entity identification apparatus connected to the user interface for receiving the disease selection information; and an display interface configured for displaying the linked graph structure and indications of identified candidate entities of interest.

In a fourth aspect, the present disclosure provides a computer-readable medium comprising code or computer instructions stored thereon, which when executed by a processor unit, causes the processor unit to perform the computer-implemented method according to any of the features of the first aspect, combinations thereof, modifications thereto, and/or as herein described.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

The features of each of the above aspects and/or embodiments may be combined as appropriate, as would be apparent to a skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1a is a flow diagram illustrating an exemplary process for identifying candidate entities of interest from search results associated with disease selection information according to the invention;

FIG. 1b is a schematic diagram illustrating an example of entity mapping for identifying entities of interest from search results associated with disease selection information according to the invention;

FIG. 1c is a flow diagram illustrating an exemplary process for identifying candidate entities of interest from search results associated with disease selection information of FIGS. 1a and 1b according to the invention;

FIG. 1d is a flow diagram illustrating an exemplary process for identifying candidate entities of interest from search results associated with disease selection information of FIGS. 1a to 1c according to the invention;

FIG. 1e is a flow diagram illustrating an exemplary process for identifying candidate entities of interest from search results associated with disease selection information of FIGS. 1a to 1d according to the invention;

FIG. 2a is a schematic diagram illustrating an example of a search query system in relation a disease selection information for identifying candidate entities of interest from search results associated with the disease selection information according to the invention;

FIG. 2b is another schematic diagram schematic diagram illustrating another search query system in relation entity selection information for identifying candidate entities of interest from search results associated with the disease selection information according to the invention;

FIG. 3 is a schematic diagram illustrating an example of a topology graph structure for identifying entities of interest from search results associated with the disease selection information according to the invention;

FIG. 4 is a schematic diagram illustrating an example of an entity mapping where predicted and known entities result sets are mapped to ontological entities of another topology graph structure according to the invention;

FIG. 5a is a schematic diagram illustrating an another example of an entity mapping system based on FIG. 2b according to the invention;

FIG. 5b is a schematic diagram illustrating an another example of an entity mapping according to the invention;

FIG. 6a is a schematic diagram illustrating another example topology graph structure where clusters of candidate entities are overlaid for identifying candidates of interest entities according to the invention;

FIG. 6b is a schematic diagram illustrating a further example of a topology graph structure where clusters of candidate entities are overlaid for identifying candidates of interest entities according to the invention;

FIG. 6c is a schematic diagram illustrating yet another example of a topology graph structure where clusters of candidate entities are overlaid for identifying candidates of interest entities according to the invention;

FIG. 7a is a schematic diagram illustrating a computing system and device according to the invention; and

FIG. 7b is a schematic diagram illustrating a system according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples. For the avoidance of any doubt, the features described in any embodiment are combinable with the features of any other embodiment and/or any embodiment is combinable with any other embodiment unless express statement to the contrary is provided herein. Simply put, the features described herein are not intended to be distinct or exclusive but rather complementary and/or interchangeable.

The present invention is related to a process and system for identifying candidate entities of interest from search results associated with, by way of example only but not limited to, disease selection information. The disease selection information may be generated upon receiving a user input associated with selecting at least one of a disease, symptoms of a disease and/or a disease process or pathway and the like. A first set of entities predicted to be associated with the disease selection information is received or retrieved, and a second set of entities that are known to be associated with the disease selection information are retrieved or received. A set of entity mappings may be generated between entities of the first set of entities, entities the second set of entities, and entities of a graph structure in relation to the disease selection information. The graph structure is based on an entity hierarchy, ontology or taxonomy of an entity family associated with the first and second sets of entities. The entities from the first and second sets of entities are linked to the graph structure based on the generated set of entity mappings. From the linked graph structure, candidate entities of interest can be identified from those linked entities of the first and second sets of entities on the graph structure based on determining where each entity from the first set of entities is located on the graph structure relative to one or more entities of the second set of entities on the graph structure.

At least following advantages of the present invention pertain to the configurations of the herein described process and system for identifying candidate entities of interest from search results associated with disease selection information. In particular, the advantage of obtaining a separate set of entities, predicted and known, is to reduce the amount of sampling required. For instance, the known set of entities provides efficient identification of a central node of a particular graph structure (may be partial graph or complete graph) as a point of reference to which parent nodes of the topology graph may, thereby reducing the amount of computation required as compared to only having a single set of entities.

In addition, the process and system as described herein enhance the reviewing of the result lists, both manual and automated, by graphically/automatically clustering entities of interest on an ontological tree /topology graph rather than scrolling linearly through a result set. More specifically, the identification of candidate entities of interest may be automated. For instance, automatic cluster recognition of one or more candidate entities may include detecting one or more clusters of entities/entities for identifying candidate entities of interest, and displaying an overlay comprising one or more indications highlighting each detected cluster of interest. In sum, the above described advantages provide improved means of reviewing search results associated with disease selection information to identify candidate entities of interest and efficiently pursuing further research and development of the identified candidate entities of interest.

A corpus of text may comprise or represent a large scale document repository including a plurality of documents, articles, literature, web-sites and/or any other digital information and/or data associated with disease selection information, entities of the first type, entities of the second type and/or entities of the second set of entities. A corpus of data or text may comprise or represent a large scale dataset or repository including any information, text or data from one or more data source(s), content source(s), content provider(s) and the like. Examples of a corpus of text used in examples according to the invention may include, by way of example only but is not limited to, documents, articles, literature, web-sites and/or any other digital information and/or data associated with disease selection information, entities of the first type, entities of the second type and/or entities of the second set of entities, unstructured data/text, one or more unstructured text, semi-structured text, documents, sections of documents, sentences and/or paragraphs of documents, tables, structured data/text, a body of text, patents and/or patent applications, publications, text, email, images and/or videos, or any other information or data that may contain a wealth of information corresponding to one or more domain(s) of interest and the like. This data may be generated by and/or stored with or by one or more sources, content sources/providers, or a plurality of sources (e.g. PubMed, MEDLINE, Wikipedia, US Patent Office databases, European Patent Office databases and/or any other patent data bases) and which may be used to form the corpus of text from which entities, entity types and entity relationships may be identified and/or extracted and the like. For example portions of text of the corpus of text (e.g. sentences, paragraphs, sections or segments of data from the corpus of text) may be retrieved and processed for identifying, detecting and/or extracting one or more entities and/or relationships thereto. A portion of text may describe an entity relationship associated with one or more entity(ies) and/or entity(ies) of interest associated with a domain of interest. The portion of text may be processed to identify, detect and/or extract, by way of example only but not limited to, a) one or more entity(ies) of interest associated with a domain of interest, each of which may be separable entities of interest; and b) one or more relationship entity(ies) that form and/or define the relationship associated with the one or more entity(ies) of interest, which may be separable.

Such large scale datasets or corpus of data/text may include data or information from one or more data sources, where each data source may provide data representative of a plurality of unstructured and/or structured text/documents, documents, articles or literature and the like. Although most documents, articles or literature from publishers, content providers/sources have a particular document format/structure, for example, PubMed documents are stored as XML with information about authors, journal, publication date and the sections and paragraphs in the document, such documents are considered to be part of the corpus of data/text. For simplicity, the large scale dataset or corpus of data/text is described herein, by way of example only but is not limited to, as a corpus of text

An entity or entity of interest may comprise or represent entity data such as, without limitation, for example an object, item, word or phrase, piece of text, or any portion of information or a fact from a portion of text and the like that may be associated with a particular entity type and be associated with a relationship. An entity or entity of interest may be, by way of example only but is not limited to, any portion of information or a fact that has a relationship, or a fact that has a relationship with another entity or entity of interest, by way of example only but is not limited to, one or more portions of information or another one or more facts and the like. An entity of interest may also comprise or represent any entity that is of interest to a user and the like. For example, in the biological, chem(o)informatics or bioinformatics domain(s) an entity of interest may comprise or represent entity data associated with an entity type from, by way of example only but is not limited to, at least the group of: gene; disease or disease process(es); compound/drug; protein; chemical, organ, biological part, tissue, cell, treatments and/or other therapies; and/or any other entity type associated with bioinformatics, chem(o)informatics, biology, biochemistry, chemistry, medicine, pharmacology, and/or any other field relevant to diagnostic, and/or drug discovery and the like. In particular, other therapies may include, by way of example, but not limited to any substance or process having therapeutic or biological impact on a target. For example, such therapeutic or biological impact may be a compound or chemotherapy affecting a gene. Other therapies may include, by way of example, but not limited to any substance or process having therapeutic or biological impact on a target. For example, such therapeutic or biological impact may be a compound or chemotherapy affecting a gene. For example, a biological entity of the biological entity type may be represented by data representative of an object, word or phrase from a portion of text that describes or is descriptive of that biological entity type based on the context of the text portion or text in which that entity resides. A biological entity may include entity data corresponding to a biological entity type associated with the biological domain based on, by way of example only but not limited to, one or more entity types from the group of: gene; disease; compound/drug; protein; chemical, organ, biological; or any other entity type associated with bioinformatics or chem(o)informatics and the like.

In general, the first set of entities may include entities predicted to be associated with the disease selection information based on any entity type from, without limitation, for example the bioinformatics and/or chem(o)informatics fields and the like. The second set of entities may include entities known to be associated with the disease selection information based on any entity type from, without limitation, for example bioinformatics and/or chem(o)informatics fields and the like. The family of entities of the graph structure may be associated with an entity type, without limitation, for example that may be represented as an entity hierarchy, ontology or taxonomy and are associated or related to the entity types of the first and second sets of entities. For example, a family of entities may be based on, without limitation, for example at least one entity type from the group of: proteins, genes, diseases and/or disease processes. The first and/or second sets of entities may be based on, without limitation, for example one or more entity types from the bioinformatics and/or chem(o)informatics fields and the like. For example, entity types from the group of: gene; disease or disease process(es); compound/drug; protein; chemical, organ, biological part, tissue, cell, treatments and/or other therapies; and/or any other entity type associated with bioinformatics, chem(o)informatics, biology, biochemistry, chemistry, medicine, pharmacology, and/or any other field relevant to diagnostic, and/or drug discovery and the like.

Each entity in the first and second sets of entities may further include data representative of entity relationships associated with the disease selection information. Entity mapping may then be performed between the first set of entities, second set of entities and the entities of the graph structure of the entity family. For example, an entity from the first set of entities may be mapped to an entity from the second set of entities based on a predicted entity relationship with the entity of the second set of entities, and vice versa. For example, the first set of entities may include one or more interacting entity pairs with a corresponding relationship therewith predicted to be associated with the disease selection information, and the second set may similarly include one or more interacting entity pairs with a corresponding relationship therewith known to be associated with the disease selection information. These interacting entity pairs and/or relationships may be used for determining entity mappings associated between each of the first set of entities, the second set of entities and the family entities of the graph structure. For instance, entity pair of the set of known interacting entity pairs may comprise a first entity of a first entity type and a second entity corresponding to an entity type associated with the entity family of the graph structure, thus the first entity of the first entity type may be mapped to the corresponding entity of the graph structure.

In one example, an entity mapping may comprise a gene that is directly mapped via a relationship onto a protein of a protein family. In another example, a compound/drug may be mapped onto a protein family via a protein, where the protein supposedly proposes the combination of known and likely drug target and protein family for the drug. In either case, the entity mapping is generated. The entity mappings that include an entity from the first or second set of entities and an entity from the graph structure may be used for linking those entities from the first or second sets of entities to the graph structure. The graph structure may be static, whereby the linking of the entities from the first and second sets of entities may use logical links, which enables the graph structure of the entity family to be reused or partially reused when further entities from the first and second sets are linked/added. The linking of entities from the first and second sets of entities to the graph structure based on the corresponding entity mappings may include both graphically and logically linking those entities from the first and second sets of entities to the graph structure, where the graphical linking may be used to visually display the graph structure and links to the entities from the first and second sets of entities, whereas the logical linking enables automatic cluster recognition to be performed to analyse the linked graph structure for automatically identifying candidate entities of interest from the first set of entities. For example, the static graph structure may comprise all protein families for a disease selection. A partial structure or a part of the graph structure (omitting parts of the graph structure that is not relevant to the disease election) may be used for purpose of displaying the relevant portion of the graph structure that includes identified candidate entities of interest. The static graph structure and maintaining a data structure including data representative of the logical links between entities of the first and second sets of entities and/or entities of the graph structure based on the entity mappings removes the need to repeatedly generate and store the topology graphs or graph structures that include all entity mappings between entities of the first and second sets of entities and/or entities of the entity family of the graph structure. This allows the effective memory management of large (increasing) data sets of predicted, known entities, and entity families and mappings therebetween.

Once entity mappings are generated based on the first set of entities, the second set of entities and/or the entity family of the graph structure, one or more entities from the first and second sets of entities may be logically linked into the entities of the graph structure of the entity family. Those entities from the first set of entities that have been mapped and/or linked onto the graph structure of the entity family, and/or linked onto entities that are also linked to the entities of the graph structure, may then be used to identify candidate entities (e.g. drugs) of interest that may lead to unique and/or useful discoveries based automatic candidate entity identification systems. Such automatic candidate entity identification systems may be based on, without limitation, for example, one or more from the group of: a set of rules based on the logical linking of the first and second sets of entities to the graph structure; one or more ML models for identifying and/or predicting candidate entities of interest based on the logical linking of the first and second sets of entities to the graph structure; and/or a hybrid system including both a rules-based system and/or ML models for identifying and/or predicting candidate entities of interest.

For example, when the graph structure is based on the disease family and the first set of entities includes entities from the drug entity type, then the set of rules may comprise, by way of example, a rule based on the shortest path and/or distance (i.e. sufficiently far) between a disease node and a node of a drug that is unprecedented for this disease or alternatively a node of a gene that is not previously discovered to be associated with the disease. Based on the set of rules, candidate entities of interest (e.g. drugs/gene/disease) may be identified as one or more clusters of entities. In particular, the entities on the topology graph are linked to a second set of entities to the graph structure based on the generated set of entity mappings. Candidate entities of interest from those linked entities of the first and second sets of entities on the graph structure based on determining where each entity from the first set of entities is located on the graph structure relative to one or more entities of the second set of entities on the graph structure may be identified. For example, a known gene and/or a predicted gene may be mapped on to the protein family based on the protein family of the gene.

The identification process and/or clustering may be further automated by automatic cluster recognition and in consideration for the purpose of an overlay that is displayed over the topology graph comprising one or more indications highlighting each detected cluster of interest. The overlay may be the display of the graph structure of an entity family to the user and overlaying the displayed graph structure with entities from the first and second sets that have been mapped to the entities of the graph structure. For instance, the entities from the first and second sets may be displayed as an overlay on the topology graph in with entities displayed in a different colour than the entities on the topology graph. This may then be used by the user in identifying candidate entities, and in an automated or semi-automated manner. The automatic cluster recognition in the case above may apply any one or more herein described ML techniques or other suitable clustering methodologies, based on training an ML model to locate regions of interest in the linked hierarchical tree structure associated with candidates of interest.

These ML techniques may include one or more or a combination of computational methods that can be used to generate analytical models, classifiers and/or algorithms that lend themselves to solving complex problems such as, by way of example only but is not limited to neural network (NN) structures, tree/graph-based classifiers, linear models and the like and/or any ML technique suitable for modelling/operating on the set of embeddings and/or an embedding vocabulary dataset generated during the training of an ML model or classifier. The trained ML model or classifier may be used to extract entities/relationships from the corpus of text. The set of embeddings and/or an embedding vocabulary dataset are generated for each of one or more relationship entity(ies) (e.g. specific relationship entities found in the text corpus describing a relationship associated with one or more specific entity(ies) of interest) with respect to the use of the ML techniques. In particular, ML techniques may be used for generating embeddings in the context of drug discovery, prediction and analysis of complex biological processes and/or related drug candidates; classification of input data in relation to one or more relationships. The training via ML techniques or the classification thereof may have the same or a similar output objective associated with input data. Data representative of the graph of entities/relationship is used as input labelled training datasets for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like.

Examples of ML techniques that may be used by the invention as described herein may also include or be based on, by way of example only but is not limited to, any ML technique or algorithm/method that can be trained on a labelled and/or unlabelled datasets to generate an embedding model, ML model or classifier associated with the labelled and/or unlabelled dataset, one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, Éclat algorithm, case-based reasoning, Gaussian process regression, gene expression programming, group method of data handling (GMDH), inductive logic programming, instance-based learning, lazy learning, learning automata, learning vector quantization, logistic model tree, minimum message length (decision trees, decision graphs, etc.), nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Naïve Bayes, hidden Markov models (HMMs), hierarchical hidden Markov model (HHMM), and any other ML technique or ML task capable of inferring a function or generating a model from labelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other an ML technique, task, or class of supervised ML technique capable of making use of unlabelled datasets and labelled datasets for training (e.g. typically the training dataset may include a small amount of labelled training data combined with a large amount of unlabelled data and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains and capable of learning or generating a model based on labelled and/or unlabelled datasets. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique capable of learning or generating a model based on learning data representations from labelled and/or unlabelled datasets.

It will be appreciated and understood by the skilled person that the ML techniques that are used to generate one or more ML model(s) as described and/or used herein may be applicable to operating on any corpus of text or literature, any type or entity type of one or more entity(ies) of interest, relationships and/or subject-matter thereto, and/or so as the application demands.

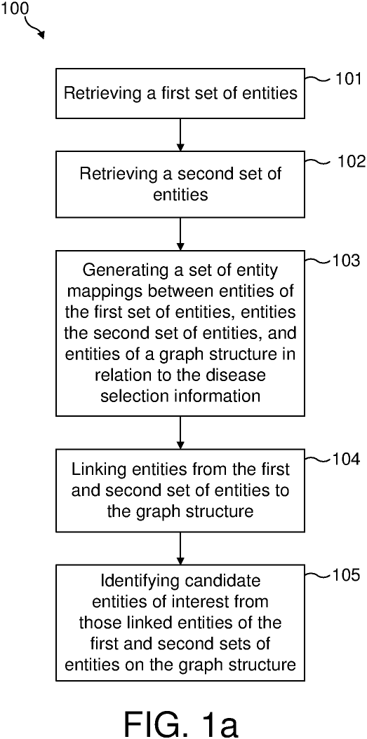

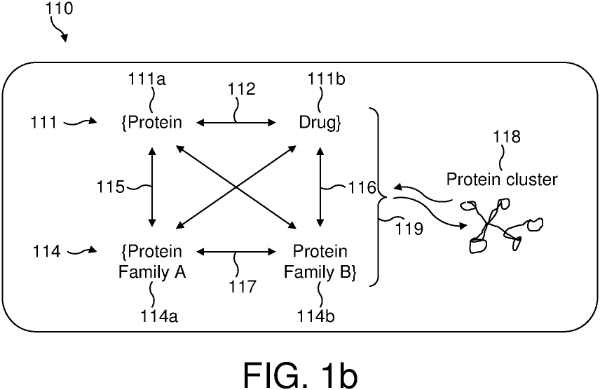

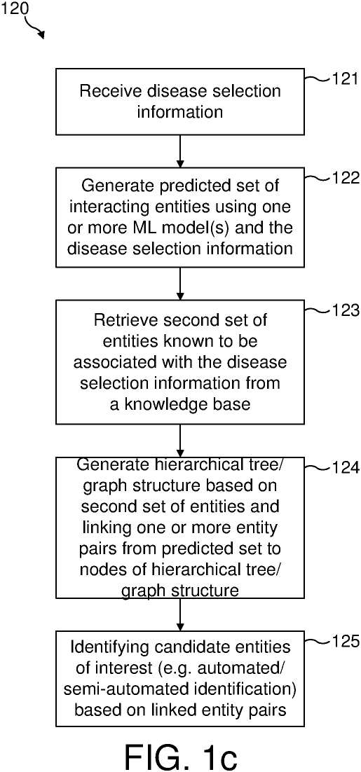

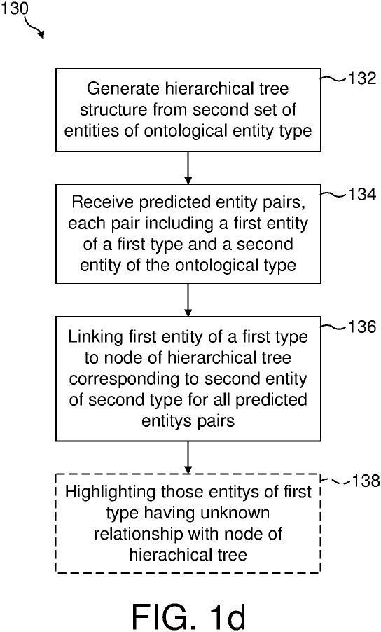

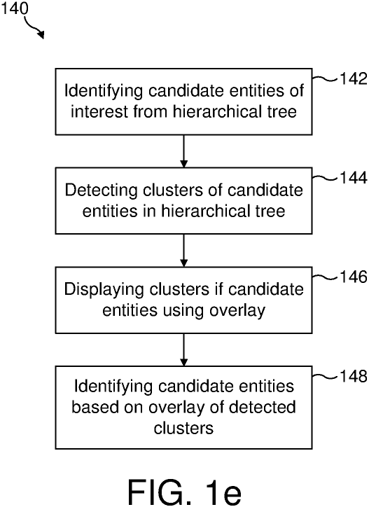

FIG. 1a is a flow diagram illustrating an exemplary process 100 for identifying candidate entities of interest from search results associated with disease selection information and/or an entity selection according to the invention. The disease selection information may include, without limitation, for example data representative of at least one from the group of: a disease, symptoms of the disease, or a disease process or pathway associated with the disease. Entity selection may include, without limitation, for example data representative of a selection of one or more entities of an entity type. The process 100 includes the following one or more steps of: In step 101, retrieving or receiving a first set of entities that are predicted to be associated with the disease selection information or entity selection (e.g. also referred to as the first set of entities (predicted)). For example, the first set of entities may be a set of entities that are predicted by one or more ML model(s) to be associated with the disease selection information or entity selection. The one or more ML model(s) may be configured to identify and/or predict entities associated with the disease selection information (or entity selection) from a corpus of text and the like. In step 102, retrieving or receiving a second set of entities that are known to be associated with the disease selection information or entity selection (e.g. also referred to as the second set of entities (known)). For example, the second set of entities may be a set of entities that may reside in a storage medium such as, without limitation, for example a database, content source(s), search index data structure, and/or knowledge base and the like and are known to be associated with the disease selection information or known to be associated with the entity selection. The second set of entities may be retrieved from the storage medium based on a set of rule(s), one or more search algorithm(s) and the like, and/or any other type of process or system for retrieving entities known to be associated with the disease selection information/entity selection. The first set of entities may include one or more entities of any entity type associated with the disease selection information (or entity selection). The second set of entities may include one or more entities of any entity type associated with the disease selection information (or entity selection).

In step 103, generating a set of entity mappings between entities of the first set of entities, entities the second set of entities, and entities of a graph structure in relation to the disease selection information (or entity selection). The graph structure may be based on, without limitation, for example an entity hierarchy, ontology or taxonomy of an entity family associated with the first and second sets of entities and/or associated with the disease selection information (or entity selection). The entity family has a plurality of entities that may be represented as a graph structure. The entity mapping may occur by combining the first set of entities (or predicted entity list) with the second set of entities (or known entity list) and then generating the graph based on these mappings. The combining may include determining the relationships each entity of the first set of entities is associated with, determining the relationships each entity of the second set of entities is associated with, and/or determining the relationships each entity of the entity family is associated with, and identifying entity mappings between the first set of entities, the second set of entities and the entities associated with the entity family. Additionally or alternatively, the entity mappings may include mapping each of the entities in the first set of entities to, if any, one or more entities of the second set of entities, and/or one or more entities of the graph structure. The mapping may be based on determining whether an entity relationship exists between each said entity of the first set of entities and each entity of the second set of entities and/or each entity of the graph structure. If a mapping exists between an entity of the first set and an entity of the second set of entities, then this entity mapping is stored in a set of entity mappings. If a mapping exists between an entity of the first set and an entity of the graph structure, then this entity mapping is stored in the set of entity mappings. If a mapping exists between an entity of the second set and an entity of the graph structure, then this entity mapping is also stored in the set of entity mappings. Each entity mapping of the set of entities may include a first entity, the entity relationship and a second entity. Each entity mapping may also indicate for each entity in the entity mapping which set of entities (predicted, known, or graph structure) said each entity belongs to. At the very least, each entity from the first set of entities in an entity mapping is indicated to be a predicted entity, and each entity from the second set of entities in an entity mapping is indicated to be a known entity. Thus a set of entity mappings may be generated based on the first set of entities, the second set of entities and the entities of the graph structure.

In step 104, linking entities from the first and second sets of entities to the graph structure based on the generated set of entity mappings. Linking those entities from the first and second sets of entities may include logically linking the entities from the first and second sets of entities to the entities of the graph structure based on the entity mappings. The data representative of the logical links may be stored separately from the data representative of the graph structure. Alternatively or additionally, the graph structure is augmented by adding the linked entities from the first and second sets of entities to the entities of the graph structure generating an augmented graph structure.

In step 105, identifying candidate entities of interest from those linked entities of the first and second sets of entities on the graph structure based on determining where each entity from the first set of entities is located on the graph structure relative to one or more entities of the second set of entities located on the graph structure. Identifying the candidate entities of interest may include automatically and/or semi-automatically identifying the candidate entities of interest based on processing the linking of entities from the first and second sets of entities to the graph structure. For example, the linked graph structure may be processed using automatic or semi-automatic identification of candidate entities of interest based on, without limitation, for example a cluster recognition system, process or engine, and/or an ML candidate identification model configured for identifying candidate entities of interest.

Step 105 may further include overlaying the linked entities on the graph structure, where overlaying may include displaying the graph structure with an overlay associated with the linked entities. Overlaying may be performed prior to identifying the candidate entities of interest, which allows a user to manually identify candidate entities of interest, and/or to assist in training an ML model to identify candidate entities of interest and the like. Alternatively or additionally, the overlaying may be performed after the system has automatically or semi-automatically identified the candidate entities of interest based on, without limitation, for example the clustering of predicted linked entities in relation to known linked entities in the graph structure, a set of rules associated with identifying candidate entities of interest from the graph structure. The candidate entities of interest may belong to the first set of entities. The overlaying may include highlighting, displaying and/or indicating the identified candidate entities of interest.

In operation, the first set of (predicted) entities and the second set of (known) entities received are used in conjunction with the entities of the graph structure to determine entity mappings between the entities of the first set of (predicted) entities, the second set of (known) entities and the entities of the graph structure. Determining these mappings may be based on the may be used to generate a graph structure, where sets of entity mappings may be derived or determined based on the associations amongst the entities of the generated graph structure. In particular, the graph structure may link entities from the first and second sets of entities based on a plurality of determined sets of entity mappings between entities of the first set of entities, the second set of entities, and family entities of the graph structure in relation to the disease selection information. With the generated graph structure, candidate entities of interest may be identified in relation to those linked entities of the first and second sets of entities based on the location/position of each entity, from the first set of entities is located in the graph structure, relative to one or more entities of the second set of entities in the graph structure. The graph structure may be based on, by way of example, but not limited to, entity hierarchy, ontology or taxonomy of an entity family associated with the first and second sets of entities.

In one example, the disease selection information may simply be a query associated with a disease. A first (predicted) set of entities received may comprise a first type and second type of entities predicted to be associated with the disease. The first type of the first set of entities may be an ontological entity (e.g. protein/gene) and the second type may be a non-ontological entity (e.g. drug/compound). The protein/gene and the drug/compound may be associated with the particular disease via an entity relationship corresponding to the protein/gene and drug/compound. In turn, a second (known) set of entities may be entities of a particular protein family, where the entities of the protein family is verified (according to a knowledge base or other trusted source) to be associated with the disease. A topology graph structure may be generated based on a gene family. One or more drugs from the first set of entities may be mapped/linked to entities of the protein families of the second set of entities based on one or more entity relationships. From the entity mappings, the topology graph structure may be augmented by linking the entities from the first and/or second sets of entities to the gene entities of the graph structure.

The resulting linked topology graph may be processed by automatic or semi-automatic identification of candidate entities of interest based on, without limitation, for example a cluster recognition system, process or engine, and/or an ML candidate identification model, and/or a user to identify, via clustering techniques, entities from the first set of entities such as, without limitation, drugs of interest that are oblivious prior to conventional tabulated result sets and the like. At the very least, entities of the first set of entities linked to the graph structure may be displayed in an overlay of the topology graph and the entities of the second set of entities linked to the graph structure may be displayed in an overlay of the topology graph to provides visual clues to a user for efficiently extract information associated with the sets of entities. The user may select a cluster of entities of the first set of entities (predicted) to be candidate entities of interest due to a close proximity and/or minimised number of hops with a cluster of entities from the second set of entities (known) linked to the graph structure, This may also be determined using an automatic clustering process, where, based on the clustering, identified candidate entities of interest are overlaid, highlighted and displayed to the user. Alternatively or additionally, identified candidate entities of interest may be stored and the result set associated with the identified candidate entities of interest sent and/or presented to the user in response to the disease selection information. The automatic/semi-automatic identification of candidate entities using, without limitation for example, automatic clustering recognition process may be based on one or more ML models that are trained based on user feedback indicating identified candidate entities of interest from the overlaid graph structure. For example, user feedback indicating user identified candidate entities of interest from the visual cues may be fed as training data for training one or more ML models to perform automatic clustering and/or identification of candidate entities of interest as herein described for purpose of further processing and identifying candidate entities of interest.

In another example, the predicted association between the ontological entity (e.g. protein/gene) and non-ontological entity (e.g. drug/compound) may constitute one or more entity pairs derived from one or more ML model(s) such as, without limitation, for example a relational model or inference model. The relational model or inference model may comprise one or more ML Model(s) herein described. The entity pair may comprise at least a protein/gene and a drug/compound and an entity relationship associated (e.g. induce/inhibit) therewith. More specifically, protein/gene and drug/compound may have a predicted relationship therewith, where the drug/compound corresponds to an entity type associated with the family entity of the graph structure. Alternatively or optionally, a protein/gene and drug/compound may have a known relationship therewith, where the drug/compound corresponds to an entity type associated with the family entity of the graph structure in accordance with the known relationship. The drug/compound may be extracted with respect to the known relationship to be included as the first (predicted) set of entities.

In yet another example, the known relationship may be used to retrieve or extract, from one or more content sources, the known set of entities. The extraction is performed on a syntactic basis when extracting context from the literature to allow more meaningful inference in, for example, a knowledge graph. Exemplary syntactic extraction are as follows:

Initial representation: (in EAT) A “IS_ASSOCIATED_WITH” (relationship label is always the same, typically “IS_ASSOCIATED_WITH”);

Representing predicates: (in SVOs) A “upregulates” B (from which we derive biological directionality: from A to B, sign: positive);

Adding context: A “upregulates” B “in liver” “in patients with ALS”.

Extracting context from the literature may further comprise: extracting anatomical location, also as biological container context, i.e. tissue, species, cell type; experimental details: e.g. assay type (the compound potency happened with a certain type of assay (e.g. cell-based)); conditional event: e.g. the activation of a particular protein, mutation of a gene; disease: this interaction is observed in patients with ALS, or in healthy patients; and gene state modifiers: e.g. phosphorylation of SOD1.

In particular, extracting context contributes to the learning of the context given a relationship. Such relationship and entities thereof may be extracted via one or more third party system(s) such as, without limitation, for example REACH, a system for automated large-scale machine reading and extraction of relationships and entities from biomedical papers; rule-based, Open Domain INformer (ODIN) and the like, one or more ML model(s) provided herein; and/or other third party systems such as, without limitation, for example, OpenIE (including the Neural version), Stanford CoreNLP, Spacy, and the like; and/or any other type of suitable system, hybrid systems combining these system(s),; combinations thereof; modifications thereto; as herein described and/or as the application demands. The extraction may be events driven, where the events are related to the relationship of a certain context. An event may be a mutation in a gene such that protein-protein interactions in the presence of mutations or drug-gene-mutation interactions may be contextually inferred during the extraction. In addition, an adaptation of generic semantic role labelling may be used to provide core arguments represent the essential arguments of a predicate, where adjunctive arguments express general properties of a predicate such as time and location. Often these adjunctive arguments contain contextual information when extracting context.

FIG. 1b is a schematic diagram illustrating an example of entity mapping 110 for identifying entities of interest from search results associated with disease selection information or entity selection according to the invention. A plurality of determined sets of entity mappings are shown in the figure. The set of entity mappings may comprise one or more mappings of: a protein 111a and a drug 111b of the first set of entities 111 via a mapping 112; a protein family A 114a and protein family B 114b of a second set of entities 114 via a mapping 117; a protein family A 114a and protein 111a via a mapping 115 or a protein family B 114b and drug 111b via a mapping 116; and mappings 119 between the protein 111a of the first set of entities 111 and the protein family A 114a of second set of entities 24. The graph structure 118 may be based on a protein family A 114a and/or B 114b to form a protein cluster or graph 118, where the entities of the set of entity mappings are linked, where applicable, to entities of the graph structure 118. In addition, the entities of the entity mapping (or mapped entities) are determined to be associated by an entity relationship therewith. Together, a protein 111a of the first set of entities 111 and a protein family A 114a of the second set of entities and an entity relationship 115 associated therewith may form an interacting entity pair, in which one entity (e.g. protein 111a) corresponds to an entity type that is the same as an entity (e.g. Protein family A 114a) of the graph structure 118. Thus, the other entity of the interacting entity pair may be linked to said same entity of the graph structure 118. Similarly, a drug 111b of the first set of entities 111 and a protein 111a of the first set of entities 111 and an entity relationship 112 associated therewith may form another interacting entity pair, in which one entity (e.g. drug 111b) corresponds to a different entity type (e.g. drug entity type) to that of the entities of the graph structure 118. But, since protein 111a forms an interacting entity pair with protein family A 114a, then it can be inferred that drug entity 111b may be linked to the entity corresponding to protein family A 114a of the graph structure 118. Thus, the drug entity 111b of the interacting entity pair of the first set of entities 111 may be linked to the corresponding entity of the graph structure 118. This procedure may be performed for all mappings 119 of the entities in the first and second sets of entities 111 or 114 and also the entities of the graph structure 118.

In operation, the entity mappings 119 are used to link entities of the first and second sets of entities 111 or 114 to the graph structure 118. Identifying candidate entities of interest in the linked graph structure 118 may be based on the positioning between the entities of the first set of entities that are linked to the graph structure 118 and those entities of the second sets of entities that are linked to the graph structure 118. Entities from the first and second sets of entities are linked as entity nodes to the graph structure 118. The position of where to link may be based on the predicted entity associations between the entity and a family entity in the graph structure 118 corresponding to the ontological, hierarchical and/or taxonomic entity family of the graph structure 118. Known associations between said each entity and a family entity in the graph structure 118 corresponding to the ontological, hierarchical and/or taxonomic entity family of the graph structure 118 may be considered or based upon when linking each entity from the first and second sets of entities as entity nodes to the graph structure 118.

In addition, when linking an entity from the first and second sets of entities to the graph structure, an indirect entity association may exist between said entity and at least one other entity of the first and second sets of entities having an association with a family entity of the graph structure. The indirect association between said entity and said entity of the graph structure are identified. The identification is based on a determined entity association between said entity and another entity of the first or second set of entities having a direct or indirect association with said family entity of the graph structure. Accordingly, the entity is linked to said another entity of the first or second set of entities.

In one example, a drug of interest may be identified to have an indirect association with a family of a protein family of interest. This indirect association may be identified based on a determined entity association between the drug and a different protein family, where the proteins amongst the family exhibit a high degree of homology.

In another example, a drug may have a direct association to known receptor, for instance, by drug mode of action. From the drug mode of action, an indirect association may be deduced for a second drug and the receptor based on known information that the second drug may be used to reduce symptoms for a disease and the fact that receptor is associated with the disease.

In a different example, the possible paths traversing the graph structure between the positioning of the linked entity pairs for the entity mappings are determined based on one or more ML model(s) herein described. More specially, the ML model(s) are configured for predicting interacting entity pairs from the first and second sets of entities associated with the disease selection information using a corpus of text as herein described. The interacting entity pairs may then be linked to the graph structure.

FIG. 1c is another flow diagram illustrating another exemplary process 120 for identifying candidate entities of interest from search results associated with disease selection information according to the invention. The process 120 may include the following steps of: In step 121, the exemplary process 120 receives the disease selection information. In step 122, the exemplary process 100 generates a predicted first set of interacting entities using one or more ML Model(s) and the disease selection information. The ML model(s) configured to predict interacting entity pairs from the corpus of text based on the disease selection information. In step 123, the exemplary process 120 retrieves second set of entities known to be associated with the disease selection information from a storage medium such as, without limitation, for example a database, content source(s) and/or knowledge base and the like. In step 124, the exemplary process 120 generates a hierarchical tree or graph structure based on an entity family associated with the disease selection information and/or based on an entity family associated with the second set of entities and linking one or more entity pairs from the predicted first set of interacting entities to nodes of hierarchical tree or graph structure.

In step 125, the exemplary process 120 identifies candidate entities of interest based on linked entity pairs. Identifying the candidate entities of interest may include, without limitation, for example using automatic candidate entities identification and/or semi-automatic candidate entity identification. The automatic and/or semi-automatic candidate entity identification may be based on, without limitation, cluster recognition of clusters of predicted entities being linked to the same, near or distant family entity node linked to a cluster of known entities. The automatic and/or semi-automatic candidate entity identification may be based on, by way of example but not limited to, ranking each of the predicted entities linked to the graph structure based on the minimum, maximum or average distance to known entities linked to the graph structure. The distance may be calculated geometrically such as, without limitation, for example, by the number of hops across or over the graph structure network, and/or other similarity measures such as, without limitation, for example ML approaches/techniques and/or ML model(s) trained based on user feedback in relation to candidate entities of interest and/or other data describing similarity or difference between different example candidate entities and/or points within the graph structure/network. Alternatively or additionally, automatic and/or semi-automatic candidate entity identification may include and employ community detection algorithms, without limitation, for example Combo, Conclude, Fast Greedy, Leading Eigen, Louvain or Spinglass for identifying clusters of predicted entities of interest, identifying clusters of known entities, and/or mixtures of both. Groups of predicted entities themselves may also be ranked based on their minimum or average distance to clusters of known entities, or the proportion of known entities making up the group. Ranked lists of entities (e.g. candidate entities of interest), or ranked groups of entities (e.g. candidate entities of interest) may be presented to users in a user interface.

In operation, the received disease selection information is used to generate the first set of entities and the second set of entities. The first set of entities may be generated by receiving a predicted set of entities output from one or more machine learning, ML, model(s) or entity identification system(s) configured for predicting or identifying entities associated with the disease selection information. The second set of entities may be generated by receiving a set of entities known to be associated with the disease selection information from one or more content source(s) or knowledge bases and the like, a content source including data representative of entities known to be associated with disease selection information. From the retrieved first and second set of entities, a graph structure based on an entity family or family type associated with an ontology, hierarchy and/or taxonomy of family entities may be generated. The graph structure comprises a plurality of entity family nodes, each entity family node representing a family entity associated with the entity family.

The graph structure is populated based on one or more entity mappings between the predicted entities of the first set of entities, between the known entities of the second set of entities, between the predicted entities and known entities of the first and second sets of entities, and/or between the predicted or known entities of the first or second sets and the family entities of the graph structure. The entity mappings are used for linking one or more entities of those entity mappings associated with an entity family node to the graph structure. Consequently, the candidate entities of interest are identified from the populated graph structure based on the positioning between the predicted entities and the known entities linked to the graph structure.

In one example, the linking or linkage is determined based on the class of relationship associated with the entity mappings, where the class of relationship may be predicted or known. The class relationship may be derived using an ML model where such a model predicts linkage based on a set of rules. Linkage may be formed between the first entity of the first type to node of the graph structure corresponding to second entity of second type for all predicted entity pairs. In effect, the entities on the graph structure are linked to a second set of entities based on the generated set of entity mappings. Candidate entities of interest from those linked entities of the first set of entities on the graph structure may be identified based on determining where each entity from the first set of entities is located on the graph structure relative to one or more entities of the second set of entities on the graph structure. In the case of a known protein and predicted gene, the known protein and/or the predicted gene may be mapped on to the protein family.

FIG. 1d is a flow diagram illustrating an exemplary process 130 for identifying candidate entities of interest from search results associated with disease selection information of FIGS. 1a to 1c according to the invention. In step 132, the exemplary process generates hierarchical tree structure from second set of entities of ontological entity type. In step 134, the exemplary process receives predicted entity pair, each pair including a first entity of a first type and a second entity of the ontological type. In step 136, linking first entity of the first type to node of hierarchical tree corresponding to second entity of second type for all predicted entity pairs. In optional step 138, highlighting those entities of the first type having unknown relationship with node of the hierarchically three.

FIG. 1e is a flow diagram illustrating an exemplary candidate identification process 140 for identifying candidate entities of interest in the graph structure using search results associated with disease selection information as described with reference to FIGS. 1a to 1d according to the invention. The candidate identification process 140 may include the following steps of: In step 142, identifying candidate entities of interest from graph structure, which may be a hierarchical tree structure. In step 144, detecting clusters of candidate entities in the graph structure (or hierarchical tree). In step 146, displaying clusters of candidate entities using an overlay. In step 148, identifying candidate entities based on overlay of detected clusters.

In operation, automatic cluster recognition (via a cluster recognition engine) may be performed to detect clusters of candidate entities of interest in relation to entities of the first set of entities and the entities of the second set of entities linked to the graph structure. The automatic cluster recognition is performed based on inputting the linked graph structure into, without limitation, for example an automatic cluster recognition ML model configured for predicting locations or regions of interest in the linked graph structure associated with candidate entities of interest. The detected clusters of candidate entities indicate the clusters of candidate entities of interest for identifying possible candidates of interest. The indicated clusters of candidate entities of interest may be displayed as an overlay over the graph structure. More specifically, the indicated clusters of candidate entities of interest display the predicted locations or regions of interest as an overlay over the graph structure. This may be used by a user or other automatic system for identifying candidate entities of interest.

Alternatively or additionally, automatic cluster recognition may be based on, without limitation, for example a rule-based system that operates on the linked graph structure based on data representative of a rule set comprising at least one or more rules from the group of: a rule associated with defining a region of interest corresponding to region of the graph structure in which an entity of the first type is linked to an entity of the second type being distant to other entities of the first type; a rule associated with defining a region of interest corresponding to a region of the graph structure in which an entity of the first type being linked to an entity of the second type, both of which have a predicted association with each other or the graph structure; a rule associated with defining a region of interest corresponding to a region of the graph structure in which the association between an entity of the first type being linked to an entity of the second type is predicted; a rule associated with defining a region of interest corresponding to a region of the graph structure in which the disease is sufficiently distant from the known treatment(s) of an existing drug; and a rule associated with defining a region of interest corresponding to a region of the graph structure in which a gene that a drug claims to be targeting is also not known to be associated with that disease.

In one example, a rule may be whether a disease being sufficiently far away from the known treatments of an existing drug/entity and therefore unprecedented. Another example of a rule may assess whether there is a lack of a classical (direct) association between the drug/entity claimed to be targeting that disease. One or more rules or the rule set may be used to identify potential candidate entity of interest.

In a further example, the ruleset for performing automatic cluster recognition may include, by way of example, but not limited to, one or more rules from a set of rules in conjunction with an ML model or other suitable clustering techniques, based on training an ML technique to locate regions of interest in the linked hierarchical tree structure associated with candidates of interest.

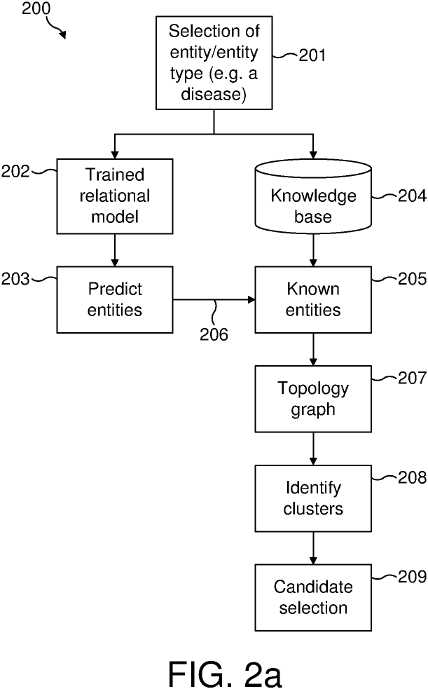

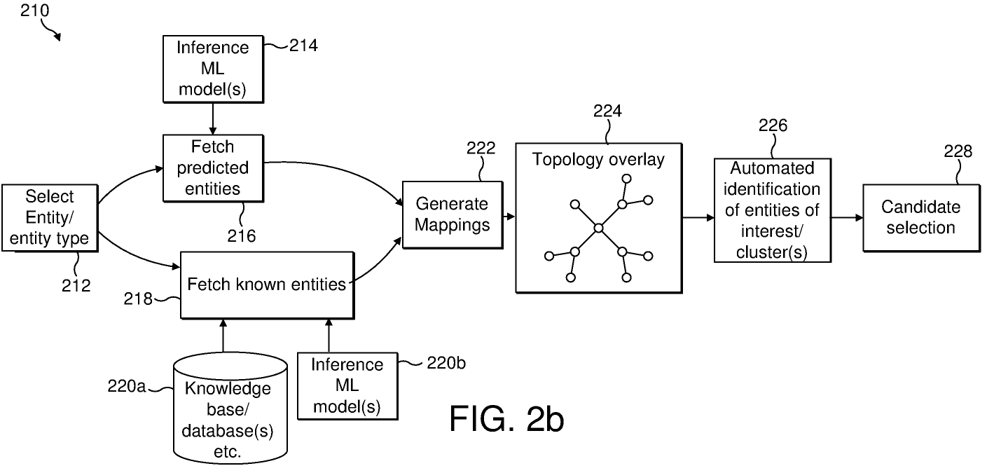

FIG. 2a is a schematic diagram illustrating an example of a system 200 for identifying candidate entities of interest in relation to selection of entity/entity type and/or disease selection information according to the invention. System 200 may be used to implement the corresponding aspects, features and/or steps of the process(es), system(s) or apparatus for identifying candidate entities of interest as described with reference to FIGS. 1a to 1e and/or 2b to 7b, modifications thereof, combinations thereof and/or as herein described. In this example, a query may be placed for a disease of interest 201 to generate disease selection information. From the disease selection information either predicted entities 203 and/or known entities 205 are produced as result sets. The predicted result set may be derived from a predicted set of interacting entities using, without limitation, for example a trained relational model 202. The trained relational model 202 may include one or more ML Model(s) that receive the disease selection information associated with the disease of interest 201 and process a corpus of text to identify and/or output a result set of entities predicted to have an association with the disease selection information. The known result set may be retrieved from a knowledge base 204 associated with the disease selection information or include other sets of entities known to be associated with the disease selection information. More importantly, the entities of the predicted result set are further mapped/linked 206 to the entities of the known result set, where the known result set may be used to produce and display a topology graph 207 based on a graph structure representing an entity family. For example, a hierarchical tree graph/structure representing a hierarchical entity ontology of the entity family. In turn, the graph structure or the hierarchical tree structure may be used to assist the cluster recognition engine and/or a user to graphically/automatically identify clusters of interest 208 in order to identify candidates of interest 209 that are not previously known to have associations with the disease of interest.

In particular, the hierarchical tree/graph structure are produced based on the known result set and linking one or more entity pairs from predicted set to nodes of hierarchical tree structure. Candidate entities of interest are thereby identified based on linked entity pairs. The hierarchical tree structure may be a graph structure based on an entity family or family type associated with an ontology, hierarchy and/or taxonomy of family entities. The graph structure may include a plurality of entity family nodes, each entity family node representing a family entity associated with the entity family.

In one example, a query is placed for a particular disease of interest to generate a list of entity types pertaining to that disease. The entity types may be used for predicting a set of protein-drug pairs associated with the disease. Similarly, a set of known protein families may be retrieved from knowledge base associated with the disease. The set of protein-drug pairs are correspondingly mapped to the protein families such that, based entity ontology, this mapping may be depicted visually through a graph structure such as, without limitation for example, a knowledge graph. The knowledge graph may highlight new candidates of interest associated with the disease that is not previously known.

FIG. 2b is another schematic diagram illustrating another example system 210 for identifying candidate entities of interest associated with the entity selection information (e.g. disease selection information) according to the invention. System 210 may be used to implement the corresponding or similar aspects, features and/or steps of the process(es), system(s) or apparatus for identifying candidate entities of interest as described with reference to FIGS. 1a to 1e and/or 2a to 7b, modifications thereof, combinations thereof and/or as herein described. System 210 includes an entity selection module 212 for receiving an entity selection information associated with an entity type (e.g. disease selection information associated with the disease entity type). The entity selection module 212 is coupled to a fetch predicted entity module 216, which is configured to output a set of entities predicted to be associated with the entity selection information. The entity selection module 212 is also coupled to a fetch known entity module 218, which is configured to output a set of entities known to be associated with the entity selection information. The fetch predicted entity module 216 and fetch known entity module 218 are both coupled to an entity mapping module 222, for generating a mapping between the set of predicted entities and the set of known entities and/or a set of family entities associated with the predicted and/or known sets of entities.

The fetch predicted entity module 216 is coupled to one or more inference ML model(s) or inference systems 214 configured for predicting and/or extracting entities from a associated with the entity selection information from a corpus of text (e.g. content or literature databases and the like associated with the entity selection information such as, without limitation, for example PubMed, patents, literature and/or any other text) to form a first set of entities predicted to be associated with the entity selection information. For example, a search query may be received by entity selection module 212 that selects a disease. This disease selection information is used by fetch predicted entities module 216 to predict and/or a set of entities output from one or more AI/machine learning, ML, model(s) 214 or entity identification/extraction system(s) 214 configured for predicting or identifying entities associated with the disease selection information from associated corpus of text. A predicted set of entities (e.g. first set of entities predicted to be associated with the entity selection information) is sent to the entity mapping module 222.

The fetch known entity module 216 is coupled to one or more knowledge base(s), databases 222a and/or from one or more inference ML model(s) or inference systems 220b configured for extracting known entities associated with the entity selection information to form a second set of entities known to be associated with the entity selection information. For example, a search query may be received by entity selection module 212 that selects a disease. This disease selection information is used by fetching known entities module 218 to retrieve a set of known entities associated with the disease selection information from knowledge bases 220a and/or from inference ML model(s) 220b and the like. A known set of entities (e.g. second set of entities known to be associated with the entity selection information) is sent to the entity mapping module 222.

The entity mapping module 222 is configured to map the predicted set of entities received from the fetch predicted entity module 216, the known set of entities received from the fetch known entity module 218, and/or entities associated with an entity family, which may be an entity family that may be represented as a graph structure (e.g. the entity family may be a hierarchical, ontological, and/or taxonomic entity family) and which is associated with the predicted and known sets of entities. For example, the disease selection information may be a disease. The predicted entities may include protein entities, drug entities, target entities and any other entity predicted to be associated with the disease. The known entities include protein entities, drug entities, target entities and any other entity known to be associated with the disease. The entity family may be selected to be a gene entity family and/or protein entity family, which is associated with the entities of the predicted and/or known sets of entities. The gene/protein entity family is an ontological entity family, so can be used to form a static graph structure. The entity mapping module may determine the relationships in relation to the predicted set of entities, the known set of entities and/or the entities of the entity family, and perform a mapping operation that determines a set of entity mappings for the one or more entities of the predicted set of entities with the known entities and/or entities of the entity family, set of mappings for the one or more entities of the known set of entities with the predicted entities and/or entities of the entity family and the like. The set of entity mappings are used to map those entities from the predicted set of entities and the known set of entities to the entities of the entity family.

The topology overlay module 224 receives the sets entity mappings and generates a topological graph with the entity family forming a static graph structure onto which those entities from the predicted and known sets of entities that may have a mapping onto one or more entities of the entity family static graph structure are linked to those entities according to the corresponding mapping. The entity nodes associated with the linked predicted and known sets of entities are topographically overlaid and displayed as linked to the corresponding family entity of the static graph structure. This may be displayed to a user and/or used or processed by an automated/semi-automated identification system 226 configured for identifying entities of interest and/or clusters of entities of interest from the predicted set of entities that are linked to the static graph structure. The automated/semi-automated identification system 226 may be implemented based on the process(es)/method(s) and/or step(s) associated with identification of candidate entities and the like as described herein with reference to FIGS. 1a to 2a and/or 3. The topography overlay may be used for identifying/selecting candidate entities of interest associated with the entity selection information and the like.

The automated identification of entities module 226 may output a set or clusters of identified candidate entities of interest to candidate selection module 228. Candidate selection module 228 may send the identified candidate entities of interest to the topological overlay module 224 for overlaying and/or highlighting the entities corresponding to the identified candidate entities of interest in the graph structure. Alternatively or additionally, the candidate selection module 228 may send or display a tabulated dataset including data representative of the identified candidate entities of interest to the user. Alternatively or additionally, the candidate selection module 228 may display a subgraph of the entity family in the regions of the entities corresponding to the identified candidate entities of interest linked to entities of the entity family.

In particular, clusters of candidate entities of interest may be identified from the displayed overlay over the graph structure or as a topography overlay, which may be determined based on a positioning between those entities of the predicted set of entities (e.g. first set of entities predicted to be associated with the entity selection information) linked to the graph structure in the overlay and those entities of the known sets of entities (e.g. second set of entities known to be associated with the entity selection information) linked to the graph structure in the overlay. For example, a cluster of several entities from the predicted set of entities linked to the graph structure that are in the vicinity of a cluster of several entities from the known set of entities linked to the graph structure may indicate a possible set of candidate entities of interest. Alternatively or additionally, the clusters of candidate entities may be determined by performing automatic cluster recognition in automated identification module 226 based on inputting the linked graph structure into an automatic cluster recognition ML model configured for predicting locations or regions of interest in the linked graph structure associated with candidates of interest. The results may be confirmed with a user and iteratively fed-back to update the ML model and/or the topography overlay. The clusters of candidate entities are displayed as a topography overlay for identifying new and previous unknown clusters for selecting candidate entities of interest. A user may intervene or interact with the generated and linked graph structure during this iterative process of updating the automatic cluster recognition and/or the topography overlay.

In operation, an automatic cluster recognition ML model for generating and updating the topography overlay is based on training an ML technique to locate regions of interest in the linked graph structure associated with candidate entities of interest. The training may be accomplished using a labelled training dataset, where the labelled training dataset for training the ML technique may be based on one or more user interactions of selecting candidate entities of interest from the multiple linked graph structures derived from multiple search queries in relation to multiple entity selection information (e.g. disease selection information). In this manner, an automatic cluster recognition system may be generated for identifying candidate entities of interest from the linked graph structure.

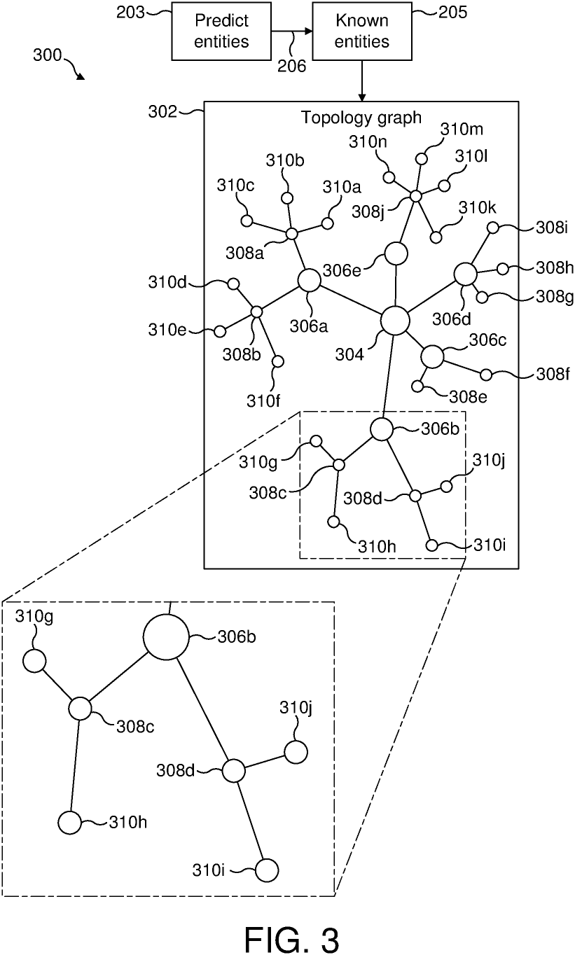

FIG. 3 is a schematic diagram 300 illustrating an example of a graph structure 302 for use in the process(es) for identifying entities of interest from entity result sets associated with the disease selection information or entity selection information as described with reference to FIGS. 1a to 2b according to the invention. The graph structure may be based on an entity hierarchy, ontology or taxonomy of an entity family associated with the first and second sets of entities. For example, the graph structure may be based on, without limitation, for example a topology graph, a hierarchical tree structure, and/or any other type of graph structure suitable for displaying an entity family. In this example, the graph structure 302 is a topology graph generated from an entity family associated with the ontological entities from the mappings 206 of the prediction entity result set 203 and the known entity result sets 205. This topology graph structure 302 may be in the form of a hierarchical tree that represents a hierarchical ontology associated with the entity family. The topology graph 302 may be displayed in a predefined format as shown in FIG. 3. In this example the topology graph 302 is shown in a splayed format. A splayed format may assist in displaying the nodes and edges between nodes of the topology graph 302. The topology graph 302 includes one or more entity family nodes, where each entity family node represents a family entity associated with the entity family or a generalised entity from the ontology. Alternatively, a visual format such as in a tabular format may be used for displaying the topology graph 302.

The central node 304 in the tree/graph 302 is the most general entity of the entity family that all parent nodes 306a/b/c/d/e representing entity subfamilies of the entity family and other child nodes 308a/b/c/d/e/f/g/h/i/j representing more specific entities may be associated and/or linked to. A parent node 306b represents a generalised entity or subfamily of the entity family or a generalised entity from the ontology. Each child node 308a/b/c/d/e/f/g/h/i/j can represent an entity subfamily of its corresponding parent node 306a/b/c/d/e or a more specific entity of its corresponding parent node 306a/b/c/d/e, with leaf nodes 310a-31 0n representing individual/specific entities associated with the entity family/subfamily etc. in the ontology.

In one example, the nodes of the topology graph 302 may represent gene/protein families. The entity subfamily may be membrane receptors that are linked to first and second child nodes representative of further subfamilies. The first child node represents the subfamily of neurotrophic factor receptors and the second child node represents the subfamily of TNF receptors. Each of the child nodes are linked to several leaf nodes. For instance, the first child node is linked to TRK family receptor and NGFR leaf nodes and the second child node is linked to Interleukin receptors and TNF family receptors.

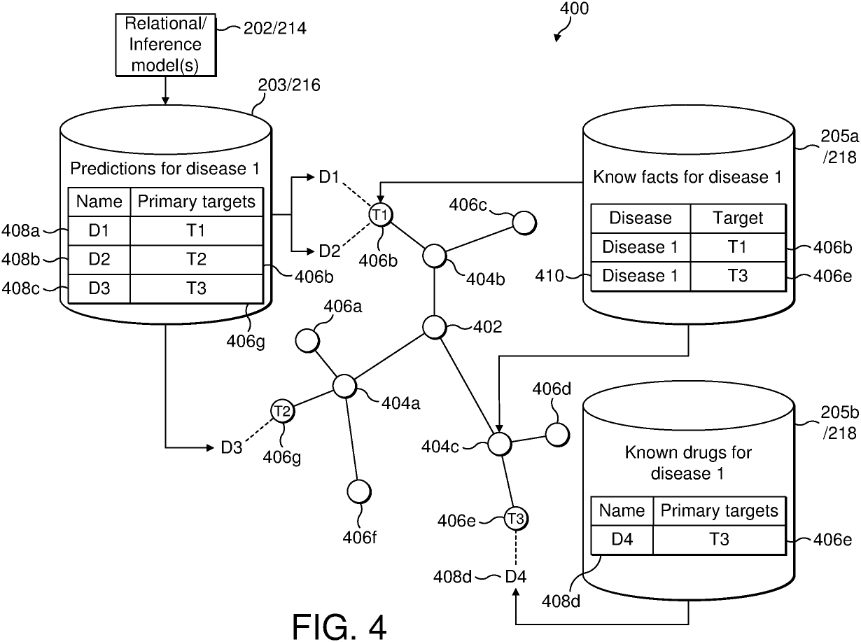

FIG. 4 is a schematic diagram illustrating an example of a graph structure 400 based on an entity family with overlays based on entity mapping and linking of predicted and known entity result sets associated with disease selection information to the entity family represented as a topology graph as described with reference to FIGS. 1a to 3 according to the invention. References to the same or similar components of FIGS. 2a and/or 2b are reused for illustration and simplicity. In this example, predicted entity result sets (e.g. first set of entities predicted to be associated with disease selection information) and known entity result sets (e.g. second set of entities known to be associated with disease selection information) are mapped and linked to ontological entities of the entity family represented as the topology graph or graph structure 400. The graph structure may be based on an entity hierarchy, ontology or taxonomy of an entity family associated with the first and second sets of entities. In this example, the graph structure 400 is based on an entity ontology of, without limitation, for example genes/proteins associated with the first and second sets of entities. The central node 402 in the graph 400 is the most general entity of the gene entity family, with parent entity family nodes 404a to 404c representing entity subfamilies of the gene entity family, with leaf entity nodes 406a-406g representing individual/specific entities associated with the gene entity family/subfamily etc. in the ontology of the gene entity family.

In a first example, a query may be made in relation to performing a search for entities of particular types such as drugs/chemicals and/or proteins/targets being associated with a particular disease 410 or disease selection information in relation to a selected entity family of a particular type such as, without limitation, genes/proteins. Although disease selection information or a disease 1 is described in this example or herein, this is by way of example only and the invention is not so limited, it is to be appreciated by the skilled person that the query may be made in relation to any entity, entity type and/or entity selection information associated with entities, entity types and the like that is of interest or as desired. Relational/inference model(s) 202/214 as described with reference to FIGS. 2a or 2b and/or as described herein may be configured to make from a corpus of text a set of predictions of entities associated with the disease 1 or disease selection information 203/216. For example, a first relational model may be configured to predict from a corpus of text a set of drug entities D1 to D3 408a/b/c that re associated with disease 1 or disease selection information, which may be output as a first subset of entities of a first type that are predicted to be associated with the disease 1 or disease selection information. Another of the relational/inference model(s) 202/214 may be configured to make another set of predictions from a corpus of text that predicted protein target entities T1 and T2 406b and 406g are associated with disease 1 or the disease selection information, which may be output as a second subset of entities of a second type predicted to be associated with the disease 1 or disease selection information. The first and second subsets of predicted entities are of different entity types, e.g. the first subset of entities is of the drug entity type and the second subset of entities is of the protein/gene target entity type. The first subset of entities 408a-408c (e.g. drugs D1-D3) may be mapped/linked to the corresponding second subset of entities 406g or 406b (e.g. targets/genes T1, T2 etc.) that they are predicted to be associated with to form the predicted entity results 203/216 of a set of predicted entity mappings from drugs to targets. In this example, the predicted entity results include predicted entity mappings of: drug D1 608a predicted to be associated with target T1 606b; drug D2 608b predicted to be associated with target T1 606b; and drug D3 606c predicted to be associated with target T2 606g. The entities associated with the predicted entity mappings of the predicted entity result sets 203/216 may be used to populate topology graph 400; and in turn, linked and/or displayed to corresponding leaf nodes that are in common with at least one entity of a predicted entity mapping of the predicted set of entity mappings. The predicted set of entity mappings are used to populate the graph 400 with drug entities associated with corresponding leaf nodes of specific targets of genes/proteins.

For example, a predicted first entity mapping of: drug D1 is associated with target T1 406b, in which the entity T1 406b has a common leaf entity node 406b of the gene entity family of graph 400. This means that from this first predicted entity mapping, the drug entity D1 408a may be linked to the entity node T1 406b of the graph 400. Similarly, a second predicted entity mapping of: drug D2 408b is associated with target T1 406b, in which the entity T1 406b has a common leaf entity node 406b of the gene entity family of graph 400. This means that from this second predicted entity mapping, the drug entity D2 408b may be linked to the entity node T1 406b of the graph 400. Finally, a third predicted entity mapping of: drug D3 408c is associated with target T2 406g, in which the entity T2 406g has a common leaf entity node 406g of the gene entity family of graph 400. This means that from this third predicted entity mapping, the drug entity D3 408c may be linked to the entity node T2 406g of the graph 400. This may assist the cluster recognition engine and/or a user for identifying candidate entities of interest.

The known result set 205a/218 is generated or fetched from known information associated with an ontological entity in relation to the disease from the knowledge base, where the underlying associations may be mapped in the graph structure 400. The known entity result set 405b may be generated or fetched based on the query for performing the search for entities being associated with the particular disease 1 or disease selection information is used to search, without limitation, for example a knowledge base, trusted repository and/or content sources for a second set of entities 205a/218 and 205b/218 known to be associated with the disease selection information. For example, a first subset of known drugs associated with the disease 1 or the disease selection information may be retrieved from a knowledge base. A second subset of known targets associated with disease 1 or the disease selection information may be retrieved from the knowledge base. The first subset of known entities may be mapped/linked to the corresponding second subset of known entities based on known associations between the first and second subsets of known entities. In this example, the known entity results include entity mappings of: drug D4 408d known to be associated with target T3 406e. The entity mappings of the known entity result sets 205a/218 and 205b/218 may be used to populate topology graph 400; and in turn, linked and/or displayed to corresponding leaf nodes and/or parent nodes that are in common with at least one entity of the known entity mapping. For example, the known entity mapping 205b/218 of: drug D4 408d is associated with target T3 406e, in which the entity T3 406e has a common leaf entity node 406e of the gene entity family of graph 400. This means that from this known entity mapping, the drug entity D4 408d may be linked to the entity node T3 406e of the graph 400. This may assist the cluster recognition engine and/or a user for identifying candidate entities of interest.

Alternatively or additionally, when the known result sets 205a/218 and 205b/218 is retrieved from a knowledge base associated with the disease selection information, the entities of the predicted result sets 203/216 may be mapped/linked to the entities of the known result set to produce one or more entity mappings or a set of entity mappings. The entity mappings may be used to produce topology graph 400 based on a graph structure or hierarchical tree structure representing a hierarchical entity ontology of an entity family associated with the predicted and/or known result sets; and in turn, displayed to assist the cluster recognition engine and/or a user to graphically/automatically identify clusters of interest in the identification of (not previously known) candidates of interest.

The linked graph structure 400 enables a user/automatic recognition (cluster) engine to understand where predicted entities linked to the graph structure 400 fit into the hierarchy of ontological entities. The known and predicted result sets 205a, 205b and 203 (or 218 and 216) are displayed in an overlay over a topology graph 400 based on a hierarchical tree structure representing a hierarchical entity ontology. In sum, the known result set 205a/218 is generated from known information associated with an ontological entity in relation to the disease from the knowledge base such that the associations may be mapped in a hierarchical fashion.

In this example, the primary targets T1 406b and T3 406e are known to be associated with the disease 1 from known facts for disease 1 410. Known drugs for disease 1 can also be retrieved and mapped drug D4 408d is known to target T3 406e. Other known result sets may also be generated from known information associated with the disease based on any other entity or entity pairs based on an ontological entity (e.g. protein/gene) and another non-ontological entity (e.g. drug/compound). The known and predicted entity result sets 205a, 205b and 203 (or 218 and 216) can be displayed as overlays on the hierarchical tree structure such a topology graph, so that a user and/or automatic recognition engine may immediately recognise relevant/irrelevant clusters of entities and focus on entities of interest. From the result set, multiple overlays may be used in conjunction with the hierarchical tree. A first overlay may be associated with relational model predictions associated with the disease (e.g. drugs D1, D2, D3 associated with targets T1, T1 and T3 respectively). A second overlay may be known facts for the particular disease (e.g. disease 1 known to be associated with target T1 and T3). A third overlay may be shown of other known facts regarding the association of entities with the particular disease or the ontological entities (e.g. drug D4 known to be associated with target T3 in relation to the particular disease).

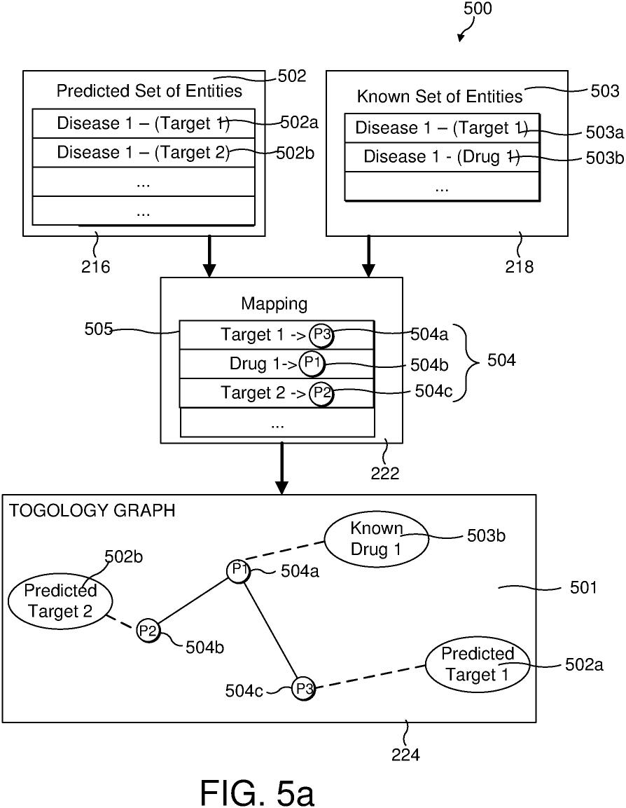

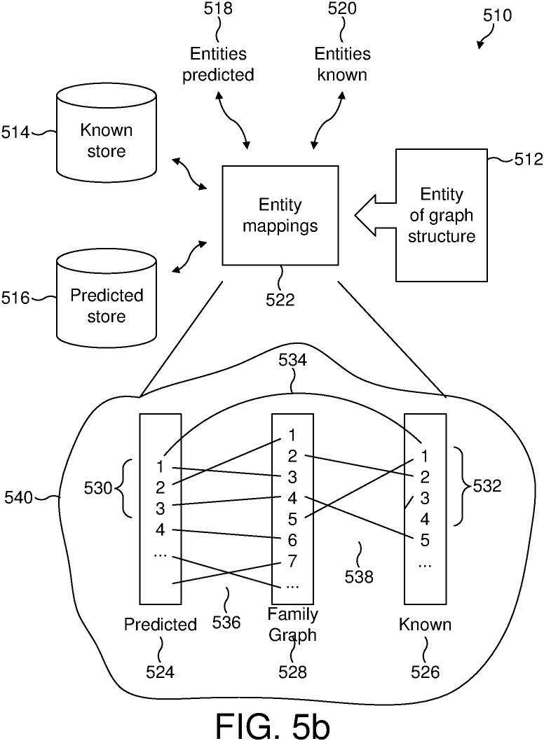

FIG. 5a is a schematic diagram illustrating another example of a graph generation system 500 for generating a topology graph structure 501 for use in identifying candidate entities of interest according to the invention. In this example, for simplicity reference numerals to similar or the same components as used in the system 210 of FIG. 2b are used. It is assumed that entity selection information has been selected in relation to an example disease 1, which is provided to fetch predicted entities module 216 and fetch known entities module 218. The fetch predicted entities module 216 uses one or more inference ML model(s) and/or rule-based systems and the like to retrieve a set of predicted entities that are predicted to be associated with the disease 1. In this case, the set of predicted entities that are associated with disease 1 include, without limitation, for example Target 1 502a, Target 2 502b and so on. Each predicted entity of the set of predicted entities 502 may include a data representative of a confidence score or ranking that provides an estimate of how reliable the predicted entity’s association with disease 1 is, i.e. the confidence that the predicted entity being associated with disease 1 is known. This is due to the predicted entity being estimated to be associated with disease 1 using inference ML model(s) over a corpus of text and the like. If there are multiple entities of the same entity being estimated to be associated with disease 1, these may be weighted and combined to form a single confidence score including data representative of a numerical value of the level of confidence that is given to the predicted entity being associated with disease 1. For example, predicted Target 502a, which is said predicted to be associated with disease 1, may have a confidence score of 0.5. Predicted Target 502b, which is said to be predicted to be associated with disease 1, may have a confidence score of 0.8. These confidence scores may be used when displaying an overlay of the known entities on the graph structure 501. The confidence score for each predicted entity may be indicative of the prediction strength associated with the predicted entity being predicted to be associated with the entity selection information (e.g. disease 1).

The fetch known entities module 218 uses one or more knowledge bases, databases, content sources, and/or inference ML model(s) and/or rule-based systems and the like to retrieve a set of known entities that are known to be associated with the disease 1. In this case, the set of known entities that are associated with disease 1 include, without limitation, for example Target 1 503a, Drug 1 503b and so on. Each known entity of the set of known entities 503 may include a data representative of a confidence score or ranking that indicates an estimate of how well known the known entity is, i.e. the confidence that the known entity is known. This may be due to the known entity being estimated using inference ML model(s) and/or from one or more knowledge bases, where there may be contradictory relationships associated with the known entity, which may be weighted etc., to form a confidence score including data representative of a numerical value of how well known or the level of confidence is given to the known entity. For example, known Target 503a, which is said known to be associated with disease 1, may have a confidence score of 0.8. Known Drug 1, which is said to be known to be associated with disease 1, may have a confidence score of 0.5. These confidence scores may be used when displaying an overlay of the known entities on the graph structure 501. The confidence score for each known entity may be indicative of the strength of conviction or a measure or estimate of how well known the known entity is known to be associated with the entity selection information (e.g. disease 1) and the like.

The sets of predicted and known entities 502 and 503 are sent to entity mapping module 222. The entity mapping module 222 also uses a selected entity family associated with the sets of predicted and known entities 502 and 503, where the entity family corresponds to a hierarchical, ontological and/or taxonomic entity family that is capable of being represented as a graph structure as described herein. In this case, the entity family may be the protein family including protein entities P1, P2, P3 and so on. Although the protein family is used in this example, this is for simplicity and by way of example only and the invention is not so limited, it is to be appreciated by the skilled person that any other type of entity family that is capable of being represented as a graph structure may be used and/or entity type as described herein that is capable of being represented as a graph structure may be used and/or as the application demands. The entity mapping module 222 may use one or more ML model(s) and/or rule-based system(s) for retrieving relationships and the like in relation to each of the entities of the predicted and/or known sets of entities 502 and/or 503 and the entities of the entity family 504. Alternatively or additionally, the ML inference models and/or knowledge bases used by modules 216 and/or 218 may also retrieve and include such information and/or relationships within the predicted and known sets 502 and/or 503. From this the entity mapping module 222 determines a set of entity mappings 505 from those entities of the predicted and known sets of entities 502 and 503 that directly and/or indirectly map onto the entities of the entity family 504.

The entity mapping may be based on combining the predicted set of entities 502 with the known set of entities 503 and then generating a graph based on these mappings. The combining may include determining the relationships each entity 502a-502b and so on of the predicted set of entities 502 is associated with, determining the relationships each entity 503a-503b and so on of the known set of entities is associated with, and/or determining the relationships each entity 504a-504c and so on of the entity family 504 is associated with, and identifying entity mappings 505 between the entities of the predicted set of entities, the entities of known set of entities 503 and/or the entities of the entity family 504.

Additionally or alternatively, the entity mappings 505 may include mapping each of the entities in the predicted set of entities 502 to, if any, one or more entities of the known set of entities 503, and/or one or more entities of the entity family 504, which make up the graph structure 501. The mappings 505 may be based on determining whether an entity relationship exists between each said entity of the predicted set of entities 502, each entity of the known set of entities 503 and/or each entity of the entity family 504 making up the graph structure. If a mapping exists between an entity of the predicted set 502 and an entity of the known set of entities 503, then this entity mapping is stored in a set of entity mappings. If a mapping exists between an entity of the predicted set 502 and an entity of the entity family 504 associated with the graph structure, then this entity mapping is also stored in the set of entity mappings. If a mapping exists between an entity of the known set 503 and an entity of the entity family 504 associated with the graph structure, then this entity mapping is also stored in the set of entity mappings. Each entity mapping of the set of entities may include data representative of a first entity, the entity relationship and a second entity. Each entity mapping may also include data representative of an indication for each entity in the entity mapping which set of entities (predicted, known, or entity family) said each entity belongs to. At the very least, each entity from the predicted set of entities 502 in an entity mapping is indicated to be a predicted entity, and each entity from the known set of entities 503 in an entity mapping is indicated to be a known entity. Thus a set of entity mappings may be generated based on the predicted set of entities, the known set of entities and the entities of the entity family 504.

In this example, it is determined that the set of entity mappings 505 include, without limitation, for example: predicted Target 2 entity 502b maps or is related to the protein entity P2 504b; predicted Target 1 entity 502a maps or is related to the protein entity P3 504c; known Drug 1 entity 503b maps or is related to the protein entity P1 504a; and so on. An indirect mapping may occur when a predicted entity maps or is related to a known entity and that the known entity maps or is related to an entity of the entity family, which means that the predicted entity maps or is related to the entity of the entity family. This mapping is also included in the entity mappings. The entity mapping module 222 then sends the set of entity mappings and/or the entity family to the topology graphing module 224.

The topology graphing module 224 generates a graph structure and/or retrieves a static graph structure based on the entity family 504. The graph structure includes a plurality of entity nodes 504a-504c linked together based on the hierarchy, ontology and/or taxonomy associated with the entity family 504. Each entity node 504a-504c is associated with an entity of the entity family 504. In this case, the entity family may be proteins, so the entity nodes are linked together based on the protein ontology or the ontological relationships associated with protein entities within the protein family. For example, the protein entity P1 504a is a parent node that is higher up in the protein ontology than protein entities P2 504b and P3 504c, which are child nodes or leaf nodes of the parent protein entity P1 504a. These are linked together by edges (represented as solid lines) to form a linked graph structure with a root parent node (e.g. node P1 504a) and a plurality of descendent nodes (e.g. nodes P2 504b and P3 504c) descended therefrom. The set of entity mappings are then used to determine which entities of the predicted set of entities 502 and which entities of the known set of entities 503 are able to be linked with edges (e.g. dashed lines) to one or more entities 504a-504c of the graph structure based on the entity family 504. Those entities from the predicted set of entities 502 and the known set of entities 503 that can be linked to entities of the entity family 504 are overlaid onto the graph structure 501 of the entity family, with entity nodes associated with the entities of the predicted and known sets of entities 502 and 503 linked by edges to the entities of the entity family. Linking the entities of the predicted set 502 and known set 503 with entities of the entity family 504 based on the entity mappings 505 may include overlaying the linked predicted and known entities on the graph structure 501 of the entity family 504. Overlaying may include displaying the graph structure 501 with an overlay associated with the linked entities from the predicted and know sets of entities 502 and 503.

In this example, given the set of entity mappings 505 include, without limitation, for example: predicted Target 2 entity 502b mapping or is related to the protein entity P2 504b; predicted Target 1 entity 502a mapping or is related to the protein entity P3 504c; known Drug 1 entity 503b mapping or is related to the protein entity P1 504a; and so on; then a predicted Target 2 entity node 502b is overlaid and linked (via a dashed line) to the protein entity node P2 504b of the graph structure 501, a predicted Target 1 entity node 502a is overlaid and linked (via a dashed line) to the protein entity node P3 504c of the graph structure 501, and a known Drug 1 entity node 503b is overlaid and linked (via a dashed line) to the protein entity node P1 504a. From the linked graph structure 501, candidate entities associated with the predicted set of entities 502 may be identified as candidate entities of interest as described, without limitation, for example with reference to automatic identification module 226 of FIG. 2b and/or as described with reference to identifying candidate entities of interest as described in process(es), method(s), steps thereof, and/or system(s) thereto with reference to FIGS. 1a to 4 and/or 5b to 7b, combinations thereof, modifications thereto, and/or the like.

Overlaying and displaying those predicted entities from the predicted set of entities 502 may be performed prior to identifying the candidate entities of interest and/or, when candidate entities of interest from the predicted set 502 are found or identified then, these may be overlaid and displayed on the graph structure 501 after identifying the candidate entities of interest. Should each of the known entities and predicted entities that are linked to an entity of the entity family have an associated confidence score, then these confidence scores may be used, when overlaying these predicted and/or known entities on the graph structure 501, to parameterise the entity node and to graphically weight or distinguish or highlight those predicted and known entities with high confidence scores over those predicted and known entities with lower confidence scores. For example, the predicted and/or known entities linked to the entities of the entity family in the graph structure 501 may be graphically weighted based on the confidence score associated with each entity from the predicted and/or known set of entities 502 and/or 503. The graphically weighting the predicated and/or known entity nodes 502a or 503b may include weighting the size, colour, shape, and other metadata associated with these entity nodes based on their corresponding confidence score.

The confidence scores may also be used when performing candidate entity identification by, without limitation, the automatic/semiautomatic candidate entity identification module 226 and/or process(es), method(s), step(s) associated with identifying candidate entities of interest as described with reference to FIGS. 1a to 4 and 5b to 7b and the like. These may assist in identifying those candidate entities of interest with the most confidence.

FIG. 5b is a schematic diagram 510 schematic diagram illustrating another example of entity mappings 522 according to the invention. In the figure, a set of entity mappings is shown between entities of the predicted set of entities 518, entities the known set of entities 520, and entities of a graph structure 512 in relation to the disease selection information is generated. The predicated and known entity may be received in real-time or from respective entity stores, known store 514 and predicted store 516 respectively. More specifically, the set of entity mappings 540 comprise an entity mapping between an entities of the predict set of entities 530, an entity mapping between an entities of the known set of entities 532, an entity mapping between an entity of the first set of entities and an entity of the second set of entities 534, an entity mapping between an entity of the predicted set of entities and a family entity of the graph structure 536, and an entity mapping between an entity of the known set of entities and a family entity 528 of the graph structure 538. In effect, the set of entity mappings links entities from the predicted and known sets of entities to the graph structure based on the generated set of entity mappings and the underlying entity relationships therewith.

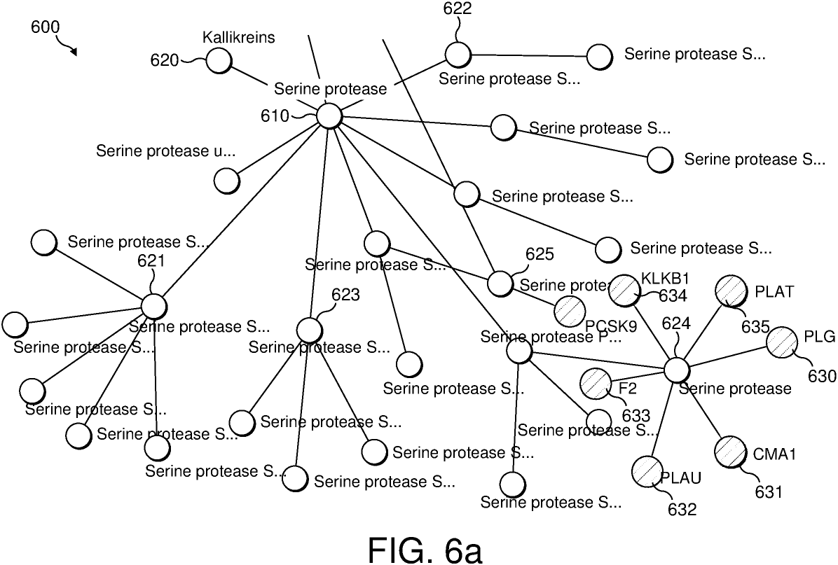

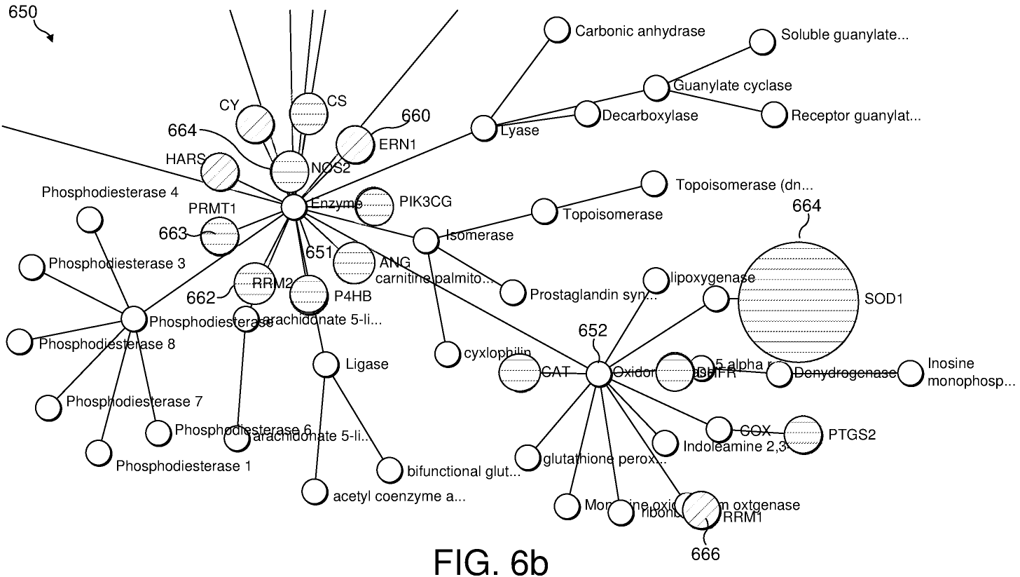

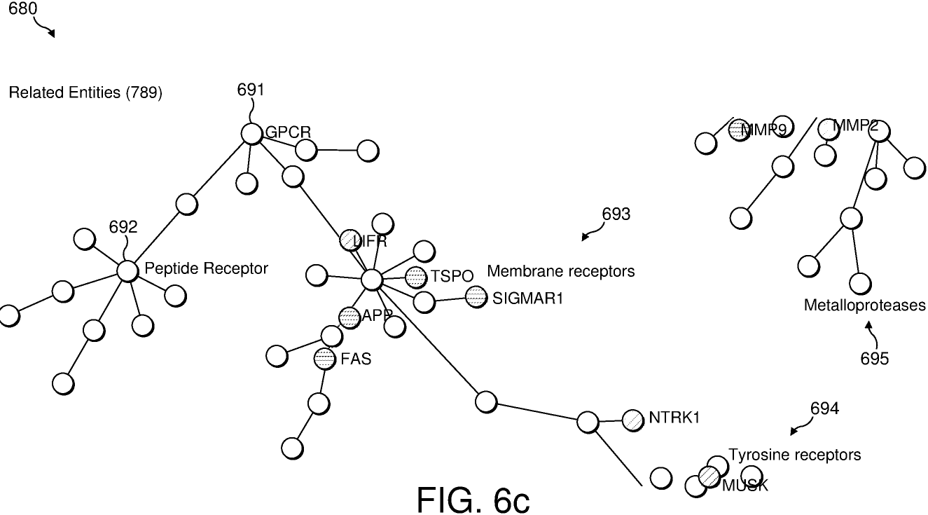

FIG. 6a is a schematic diagram illustrating an example of another topology graph 600 where clusters of candidate entities are overlaid for identifying candidates of interest entities according to the invention. The topology graph 600 depicts clusters of entities of interest (entity nodes with hash marks) that are overlaid and displayed on the topology graph 600 of an entity family associated with serine protease, which are represented as a plurality of entity family nodes including the serine protease entity nodes (610, 620, 621, 622, 623, 624, 625) represented as white circles with black outline. The serine protease entity nodes: 620, 621, 622, 623, 624 and 625 are subfamily/subgroup entity nodes that relate to a particular (serine) protease of interest at the central node 610. Each entity family node (610, 620, 621, 622, 623, 624, 625) of the topology graph is a protease of the family of the serine protease that may be involved in various biological processes. For instance, Kallikreins node 620 (often known for such physiological function as regulating blood pressure) is an entity family node 620 that is a subgroup of serine proteases that are related to the serine protease family (S1A family) 610 at the central/root node, which is shown as an entity pair on based on the ontology of the serine protease entity family. Similarly, different subfamilies of serine proteases are distributed across various entity family nodes (620, 621, 622, 623, 624, 625) of the topology graph 600. Different families of serine proteases are arranged in the topology graph structure 600 based on ontology with the more prominent relationship being closer in distance with respect to the serine protease node 610 as the central/root node 610. The distance may be based on the minimum or average distance to each entity of interest, with distance being calculated geometrically, by the number of hops traversing the topology graph.

In particular, serine protease S1A family are endopeptidases that cleave peptide bonds (proteolysis) in which serine serves as the nucleophilic amino acid at the active site. In FIG. 6a, the designated “Serine Protease S...” entities, on the topology graph 600, that stem from the serine protease node 610 as the central/root node 610 of the S1A family are related to Elastase-Like and serine protease 6. For instance, the group may include, by way of example, but not limited to specific gene member: SP46, SP52, SP60, BDNA: GH08420, SP90, SP151, Ser 4, SP137, Jon25B, SP 112, Jon65A, SP98, SP 171, Jon66C, Jonah 66c, Ser99Dc, Jon99C, Jon 99Cβ, Jon99Cβi, SER3, and SP47. In addition, other members such as SP 154, SP 185, and SP189 may be associated with component group of Chymotrpsin-like and Trypsin-like serine proteases, where the member may be a specific member or groups of member of the S1A family.

Furthermore, FIG. 6a illustrates the overlay of various gene entities as gene entity nodes 630, 631, 632, 633, and 634, which are represented as circles with diagonal hash lines. In general, the overlay links predicted and known entities of different entity types on the graph structure associated with the entity family, whereby overlaying comprise displaying the graph structure of the entity family with an overlay associated with the linked entities in order to identify the candidate entities of interest. In FIG. 6a, the gene entities include PLG, CMA1, PLAU, F2, KLKB1, PLAT and PCSK9 (e.g. displayed as hashed circles) encoding respective proteins are found (e.g. known or predicted entities) and mapped to corresponding serine protease subfamily entity nodes and linked to the topology graph structure of serine protease 600. In this case, the gene entities include PLG, CMA1, PLAU, F2, KLKB1 and PLAT are represented as gene entity nodes 630, 631, 632, 633, 634 and 635 that are linked and associated with the serine protease p. node 624. The edges direct the relationship between the corresponding gene entity node and the serine protease entity 624. The overlay provides at least a visual cue for identifying clusters of gene entities and hence candidate entities of interest. In particular, the identification of the candidate entities of interest is accomplishing by ranking the predicted entities based on the minimum or average distance to known entities, with distance being calculated geometrically, by the number of hops traversing the network/graph structure, or other similarity measures/approaches, given user feedback or other data describing similarity or difference between different example points within the graph structure.

FIG. 6b is a schematic diagram illustrating an example of another topology graph 650 where clusters of candidate entities are overlaid for identifying candidate entities of interest according to the invention. In this example, a family entity set of entity nodes form a static graph structure of the topology graph 650 represented by the nodes with white circles and black outlines, with edges linking the taxonomy, hierarchy, or ontology representing the family entity set together. The predicted set of entities (e.g. first set of entities) mapped onto the topology graph 650 are represented by predicted entity nodes, which are represented as horizontal hashed nodes, with the edges between an entity node of a predicted entity and another family node of the topology graph representing the relationship between the predicted entity and the other family node of the topology graph 650 based on the mapping thereto. The known set of entities mapped onto the topology graph 650 are represented by the known entity nodes, which are represented as diagonally hashed nodes, with the edges between an entity node of a known entity and another family node of the topology graph 650 representing the relationship between the known entity and the other family node of the topology graph 650 based on the mapping thereto.

In the part of the topology graph 650 linking predicted and known entity nodes to the family entity nodes of the static graph of the topology graph 650, the predicted and known entity nodes are represented based on size, colour, shading, hashing, and/or shape properties to distinguish between the predicted entity nodes (entities) and known entity nodes that are either in the first (predicted) or second (known) set of entities respectively. That is, entity nodes are represented differently in relation to size, colour, hashing and/or shape properties to emphasise those entity nodes that are family entities (e.g. white nodes with black circles) making up a static graph part of the topology graph 650, those entity nodes of the known set of entities mapped to the family entities (e.g. nodes with diagonal hashes) and/or predicted entities, and those entity nodes of the predicted set of entities (e.g. nodes with horizontal hashes) mapped to the family entities and/or known entities. For example, the size of an entity node may represent, by way of example only but not limited to, prediction strength for those entity nodes (entities) corresponding to the predicted set of entities mapped known set of entities and/or family sets of entities. Prediction strength can be measured, without limitation for example, as an aggregate of a number of predictions or relationships extracted or inferred that are identical or aggregated to have the same meaning. Alternatively or additionally, prediction strength can be based on, without limitation, for example the confidence or confidence score of the ML model that predicted the relationship, since a number of ML models might be used to infer and/or extract relationships from the corpus of text in relation to the predicted set of entities. In particular, known entities (nodes with diagonally hashed lines) and predicted entities (nodes with horizontally hashed lines) are overlaid or represented in an overlay with those family entities that are (embedded) as part of the static graph structure of the topology graph 650 herein displayed. In the overlay there are enlarged nodes with sizing being dependent on prediction strength and/or confidence score attributed to the entity prediction from the ML model etc. In this case, the predicted gene entity SOD1 664 has a higher prediction strength/confidence score than prediction entity RRM2 662, predicted entity PRMT1 663, or predicted entity NOS2 664, while known entity ERN1 660 is a known entity.

In an example, the prediction strength/confidence score may be assessed based on how many connections may be predicted from the data, and how well. For instance, prediction strength/confidence score may take the minimum of the proportion of observation pairs in that cluster that are also assigned to the same cluster over the k test clusters. Predicted entity NOS2 664 could be further distinguished from predicted entity PRMT1 663 based on distances from the Enzyme family node 651 as the central node 651 suggesting the proximity of the relationship therewith. Entity nodes PRMT1 663, ERN1 660, and NOS2 664 are linked to a central node (Enzyme) 651 of this partial graph 650. The central nodes may vary between graphs or depending on what part of the graph being considered, i.e. oxidoreductase 652 may be a central node in cases where the disease selection information relates to oxidoreductase.

In particular, the distances (or relative distances) distinguishing the entity NOS2 664 from PRMT1 663 may be, for example, the minimum or average distance. The minimum or average distances may be calculated geometrically between enzyme 651 and respectively NOS2 664 and PRMT1 663. The distances are assessed based on the number of hops traversing the topology graph 650 from a predicted entity node to another. The distances may be measured using algorithms or via herein described ML approaches, given user feedback or other data describing similarity or difference between different example points within the topology graph.

Moreover, the nodes may also comprise their associated metadata (derivable from itself or from other nodes) to which may contribute to the prediction strength, for example, metadata in response to a change in connectivity of the graph. In particular, the metadata may be associated with the connector operator of a particular node that may encode the type of connection. Metadata may be parameters associated with input and output of the nodes and how they are being linked or added/updated onto the graph structure.

FIG. 6c is a schematic diagram illustrating an example of yet another topology graph 680 where clusters of candidate entities are overlaid for identifying candidates of interest entities according to the invention. The entity family is based on the protein family and the predicted and known entities are based on gene entities. A partial graph is shown of the full graph of 789 related entities generated based on a set of entity mapping between known gene entities in relation to the disease selection information, predicted gene entities in relation to disease selection information, and/or the protein entity family entities/groups/subgroups associated with the protein entity family. The entity family nodes of the protein entity family associated with the graph 680 are represented by white nodes with black outlines. The known gene nodes that are linked to the entity family graph 680 are represented by nodes with diagonal hashes. The predicted gene nodes that are linked to the entity family graph 680 are represented by nodes with horizontal hashes.

In this example, the protein entity graph or topology graph 680 is overlaid with 4 known genes (i.e. NTRK1, MUSK, MMP2, LIFR) are shown and 5 predicted genes (APP, FAS, TSPO, SIGMAR1, MMP9). The known genes and predicted genes are overlaid onto the various protein families corresponding to 41 nodes associated with proteins of the respective protein families underlying the set of entity mappings. These protein families include, in general, G-protein-coupled receptors (GPCRs) 691, Peptide receptors 692, Membrane receptors 693, Tyrosine protein 694, and Metalloproteases 695. On the other hand, the predicted and known genes are linked so as to allow the display of the graph structure with an overlay associated with the linked genes. The displayed graph structure 680 as such is generated and updated based on an entity hierarchy, ontology or taxonomy of an entity family associated with the known and predicted sets of genes herein presented, which in this case is the protein entity family.

As described herein with reference to FIGS. 1a to 6c and/or 7a to 7b , one or more ML models may be used for predicting entities for the first set of entities that are predicted to be associated with the disease selection information and/or entity selection information, and/or predicting entities for the second set of entities known to be associated with the disease selection information or entity selection information. For example, a machine learning (ML) relational or inference model may be used, where the ML relational/inference model may be trained using labelled training datasets and/or rulesets for determining entities associated with disease selection information or the pairs of interacting entities associated with disease selection information from one or more corpus of text pertaining to such field as biology, biochemistry, chemistry, medicine, chem(o)informatics, bioinformatics, pharmacology, and any other field relevant to diagnostic, treatment, and/or drug discovery. The ML relational/inference model(s) may determine, in this case, whether the portions of text of a text corpus includes at least one or a pair of interacting entities in accordance with the disease selection information as input. Based on this determination, entities and/or interacting entity pairs may be retrieved from the corpus of text as to generate a predicted set of entities as output.

In one example of the disease election information may be Alzheimer’s with the symptoms of memory loss. Given the disease selection information, the ML relational model predicts a set of genes-protein entity pairs such as APP-β-amyloid peptide, associated with Alzheimer’s provided that the ML model is trained via labelled training datasets and/or rulesets for determining pairs of interacting entity associated with disease selection information. The dataset and/or rulesets may be based on dataset describing similarity or difference between different example points within the network/graph structure, subject to user feedback. A ranking of predicted entities may be produced based on the minimum or average distance to known entities, with distance being calculated geometrically, by the number of hops traversing the graph structure.

As described herein with reference to FIGS. 1a to 6c and/or 7a to 7b, one or more ML models may be used for identifying candidate entities of interest from a linked graph structure. For example, an ML candidate entity identification model or an automatic cluster recognition ML model according to the invention may be used to process the linked graph structure as described herein to identify candidate entities of interest, which may be overlaid on the graph structure and/or displayed on the graph structure to a user. In general, automatic cluster recognition identifies candidates of interest by separating input of candidate entities of interest into clusters of candidate entities of interest. The clusters of candidate entities may be displayed (not shown) as an overlay over the graph structure of an entity family. In particular, the candidate entity identification or automatic cluster recognition ML model(s) may predict locations or regions of interest in the linked graph structure associated with candidates of interest by locating regions of interest in the linked graph structure associated with candidates of interest using a labelled training dataset, where the labelled training dataset used by the ML technique for training the ML model is based on one or more user interactions of selecting candidate entities of interest from the multiple linked graph structures derived from multiple search queries in relation to multiple disease selection information/entity selection information or from a rule set associated with locating regions of interest in the linked graph structure in relation to candidate entities of interest may be used. In one supervised example, the candidate entity identification or automatic cluster recognition ML model uses the labelled training dataset to estimate for a specific label based on a k-dimensional input of candidates of interest. This task is accomplished by using the rule set associated with locating regions of interest that is able to predict the label of new patterns. Effectively, the rule set may be updated or adjusted accordingly, given no further constraint imposed upon the specific label space. The candidate entity identification or automatic cluster recognition ML model thereby estimate clusters of candidates of interest for the specific label. In one example, entities of the topology graph are taken as input into the candidate entity identification or automatic cluster recognition ML model where k-means clustering is applied for the assignment of the data members to k number of clusters, or groupings for the entities. Based on the grouping, entities of interest may be identified in accordance with the number of clusters from the underlying entities.

Additional community detection algorithms may be used for identifying clusters of predicted entities, known entities, or both, together with or in place of the ML model herein described. These algorithms include, by way of example, but not limited to Combo, Conclude, Fast Greedy, Leading Eigen, Louvain, and Spinglass. Whether using one or more algorithms or otherwise using one or more ML models, to group the entities, for which grouping may be further ranked based on their minimum or average distance to clusters of known entities, or the proportion of known entities making up the group. Accordingly, ranked lists of entities, or ranked groups may be presented to users on a user interface, where the user interface may display and/or identify areas of user’s interest. Such identification may be achieved using one or more algorithms or via one or more ML model described herein.

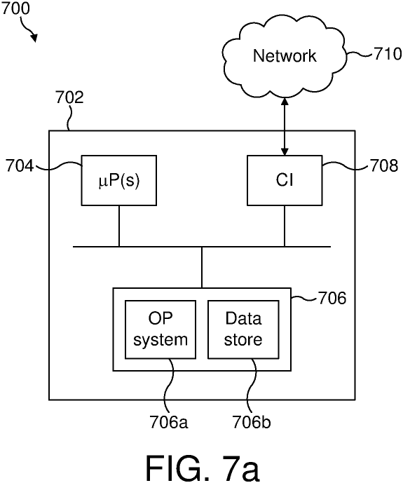

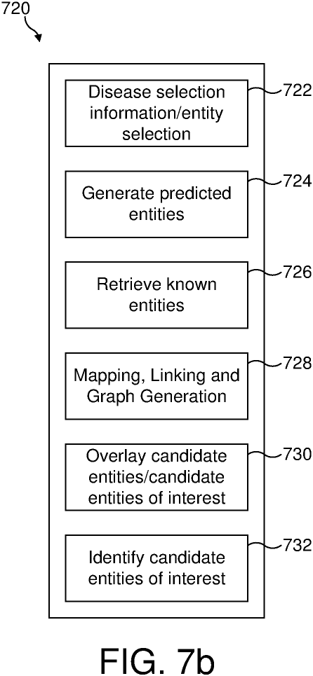

FIG. 7a is a schematic diagram illustrating a computing system 700 including a computing device, server and/or apparatus 702 coupled to a communications network 710 that may be used to implement one or more aspects of the process(es), system(s), method(s) ML model(s) and the like according to the invention and/or implement one or more aspects of the process(es), system(s), method(s) and/or ML model(s) and apparatus as described with reference to FIGS. 1a to 6c and/or 7b, combinations thereof, modifications thereto, herein described and/or as the application demands. Computing device 702 includes one or more processor unit(s) (µPs) 704, memory unit 706 and communication interface (CI) 708 in which the one or more processor unit(s) 704 are connected to the memory unit 706 and the communication interface 708. The communications interface 708 may connect the computing device 702 over communication network 710 with one or more databases, corpus of text and/or other processing system(s) or computing device(s)/server(s). The memory unit 706 may store one or more program instructions, code or components such as, by way of example only but not limited to, an operating system (OS) 706a for operating computing device 702 and a data store (DS) 706b for storing additional data and/or further program instructions, code and/or components associated with implementing the functionality and/or one or more function(s) or functionality associated with one or more of the method(s) and/or process(es) of the apparatus, module(s), ML model(s), systems(s), mechanisms and/or system(s)/platforms/architectures as described herein and/or as described with reference to at least one of FIGS. figure(s) 1a to 7b.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es), modifications thereto, and/or combinations thereof as described herein with reference to any one of FIGS. 1a to 7b.

FIG. 7b is a schematic diagram illustrating a system 720 according to the invention. The system may include a query module or component 722 for receiving a disease selection information and/or entity selection and the like and forming a query accordingly; a predicted entity generation module 724 for generating a set or list of predicted entities based on the query; a known entity fetching module 726 configured for retrieving a set or list of known entities based on the query; a graph or topology graph generation module 728 configured for generating data representative of a graph structure associated with an entity family in relation to the predicted and known sets of entities, for mapping the predicted and known sets of entities to each other and the entity family and the like, for linking those predicted and known mapped entities to each other and/or the entities of the family graph; an overlay module 730 configured for displaying the family graph overlaid with those entities from the predicted and known sets of entities linked to the family graph and the like, the overlay module may display the predicted entities based on prediction confidence and distinguish these from the known entities linked to the graph, the overlay module may display and/or highlight/distinguish identified candidate entities of interest based on results from the identification module 732 and the like; and an identification module 732 configured for identifying candidate entities of interest based on the locations of the predicted entities in relation to the known entities linked in the graph, the identification may be based on automated and/or semi-automated candidate identification algorithms based on identifying clusters of predicted entities linked to the graph that are likely to include candidate entities of interest, the candidate entities of interest; The system 720 and modules/components 722-732 may include the functionality of the method(s), process(es), and/or system(s) associated with the invention as described herein, or as described with reference to FIGS. 1a-7a, combinations thereof, modifications thereto and/or as the application demands and the like.

The system 720 may include a user interface configured for receiving data representative of a query such as disease selection information or entity selection and the like; a candidate entity identification apparatus connected to the user interface for receiving the query based on disease selection information/entity selection, where the candidate entity identification apparatus generates predicted entities and retrieves known entities associated with the received disease selection information. The predicted entities and known entities are mapped and linked to a graph structure, the graph structure based on a family of entities associated with the predicted and/or known entities. The system 720 may further include a display interface configured for displaying the linked graph structure with candidate entities of interest and indications of identified candidate entities of interest highlighted or overlaid on the graph structure. In one example, the user interface may be a graphic user interface (GUI) or an application to be shown in a destination display context. Multiple elements of the GUI based on the display context and input parameters for the GUI may be assessed according to the measure of priority associated with respective ones of the elements. In another example, the display interface may be of liquid crystal display that uses the light-modulating properties of liquid crystals combined with polarizers.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es); modifications thereof; combinations thereof; as described herein; and/or as described with reference to FIGS. 1a to 9b.

In the embodiment(s) described above the method(s), apparatus, system(s) and/or computing system/device(s) may be implemented by a server, the server may comprise a single server or network of servers. In some examples the functionality of the server may be provided by a network of servers distributed across a geographical area, such as a worldwide distributed Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es); modifications thereof; combinations thereof; as described herein; and/or as described with reference to FIGS. 1a to 7b.

In the embodiment(s) described above the method(s), apparatus, system(s) and/or computing system/device(s) may be implemented by a server, the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic or semi-automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Application Program-specific Integrated Circuits (ASICs), Application Program-specific Standard Products (ASSPs), System-on-a-chip systems (SOCs). Complex Programmable Logic Devices (CPLDs), etc.

Although illustrated as a single apparatus or system, it is to be understood that the computing device or system may be a distributed system or part of a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface). Furthermore, the systems, apparatus, and/or method(s) as described herein may be distributed or located remotely and accessed via a network or other communication link (e.g. using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “module”, “component” and/or “system” are intended to encompass computer-readable data storage that is configured with computer-executable instructions that cause certain functionality to be performed when executed by a processor. The computer-executable instructions may include a routine, a function, or the like. It is also to be understood that a module, component and/or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims. Although various embodiments have been described above with a certain degree of particularity, or with reference to one or more individual embodiments, those skilled in the art could make numerous alterations to the disclosed embodiments without departing from the spirit or scope of this invention.

1. A computer-implemented method of identifying candidate entities of interest associated with disease selection information, the method comprising:
receiving a first set of entities that are predicted to be associated with the disease selection information;
retrieving a second set of entities that are known to be associated with the disease selection information;
generating a set of entity mappings between entities of the first set of entities, entities of the second set of entities, and entities of a graph structure in relation to the disease selection information, the graph structure based on an entity hierarchy, ontology or taxonomy of an entity family associated with the first and second sets of entities;
linking entities from the first and second sets of entities to the graph structure based on the generated set of entity mappings; and
identifying candidate entities of interest from those linked entities of the first and second sets of entities on the graph structure based on determining where each entity from the first set of entities is located on the graph structure relative to one or more entities of the second set of entities on the graph structure.

2. The computer-implemented method of claim 1, further comprising: overlaying the linked entities on the graph structure, wherein overlaying comprises displaying the graph structure with an overlay associated with the linked entities.

3. The computer-implemented method as claimed in claim 1 further comprising determining sets of entity mappings based on determining entity associations between entities of the first set of entities, entities of the second set of entities, and/or family entities of the graph structure.

4. The computer-implemented method according to claim 1 , wherein the set of entity mappings further comprising any one or more entity mappings from the group of:
an entity mapping between an entities of the first set of entities, wherein the entities of the entity mapping are determined to be associated by an entity relationship therewith;
an entity mapping between an entities of the second set of entities, wherein the entities of the entity mapping are determined to be associated by an entity relationship therewith;
an entity mapping between an entity of the first set of entities and an entity of the second set of entities, wherein the entities of the entity mapping are determined to be associated by an entity relationship therewith;
an entity mapping between an entity of the first set of entities and a family entity of the graph structure, wherein the entities of the entity mapping are determined to be associated by an entity relationship therewith; and
an entity mapping between an entity of the second set of entities and a family entity of the graph structure, wherein the entities of the entity mapping are determined to be associated by an entity relationship therewith.

5. The computer-implemented method as claimed in claim 1, wherein identifying candidate entities of interest further comprises identifying candidate entities of interest in the graph structure based on a positioning between those entities of the first set of entities added to the graph structure and those entities of the second sets of entities added to the graph structure.

6. The computer-implemented method according to claim 1, wherein linking further comprising:
adding each entity from the first and second sets of entities as entity nodes to the graph structure based on predicted entity associations between the entity and a family entity in the graph structure corresponding to the ontological, hierarchical and/or taxonomic entity family; and
adding each entity from the first and second sets of entities as entity nodes to the graph structure based on known associations between said each entity and an family entity in the graph structure corresponding to the ontological, hierarchical and/or taxonomic entity family.

7. The computer-implemented method as claimed in claim 1, further comprising graphically weighting each entity node linked to the graph structure based on a confidence score associated with each entity from the first and/or second set of entities, wherein the graphically weighting may include weighting the size, colour, shape, and other metadata associated with the entity nodes based on the corresponding confidence score.

8. (canceled)

9. The computer-implemented method as claimed claim 1, wherein adding an entity from the first and second sets of entities to the graph structure further comprises adding said entity to the graph structure as an entity node when an indirect entity association exists between said entity and at least one other entity of the first and second sets of entities having an association with an family entity of the graph structure, the method further comprising:
identifying an indirect association between said entity and said entity of the graph structure based on a determined entity association between said entity and another entity of the first or second set of entities having a direct or indirect association with said family entity of the graph structure, and
linking the entity by adding it to said another entity of the first or second set of entities.

10. (canceled)

11. The computer-implemented method as claimed in claim 1, wherein the disease selection information comprises data representative of at least one from the group of: one or more diseases, one or more symptoms of the one or more diseases, one or more cell types associated with the one or more diseases, one or more tissue types associated with the one or more diseases, one or more organs associated with the one or more disease, one or more biological parts associated with the one or more diseases, or one or more disease processes associated with the one or more diseases, and
wherein an entity comprises entity data associated with an entity type from at least the group of: gene; disease or disease process(es); compound/drug; protein; chemical, organ, biological part, tissue, cell, treatments and/or other therapies; and/or any other entity type associated with bioinformatics, chem(o)informatics, biology, biochemistry, chemistry, medicine, pharmacology, and/or any other field relevant to diagnostic, and/or drug discovery and the like.

12. (canceled)

13. The computer-implemented method as claimed in claim 1 wherein:
the first set of entities are associated with an entity type from the group of: diseases, disease process(es) and the like;
the second set of entities are associated with an entity type from the group of: drugs, chemicals, compounds, pharmacology, treatments and/or other therapies and the like; and
the family entities of the graph structure are associated with an entity type from the group of: proteins, genes, diseases and/or disease processes.

14. The computer-implemented method as claimed in claim 1 wherein:
receiving the first set of entities further comprises receiving a predicted set of entities output from one or more machine learning, ML, model(s) or entity identification system(s) configured for predicting or identifying entities associated with the disease selection information;
retrieving the second set of entities further comprises receiving the second set of entities from retrieving entities known to be associated with the disease selection information from one or more content sources, a content source including data representative of entities known to be associated with disease selection information;
generating the graph structure further comprises generating the graph structure based on an entity family or family type associated with an ontology, hierarchy and/or taxonomy of family entities, wherein the graph structure comprises a plurality of entity family nodes, each entity family node representing a family entity associated with the entity family;
adding entities to the graph structure further comprises populating the graph structure based on one or more entity mappings between the predicted entities of the first set of entities, between the known entities of the second set of entities, between the predicted entities and known entities of the first and second sets of entities, and/or between the predicted or known entities of the first or second sets and the family entities of the graph structure, and linking one or more entities of those entity mappings associated with an entity family node to the graph structure; and
identifying candidate entities of interest from the populated graph structure based on the positioning between the predicted entities and the known entities added to the graph structure.

15. The computer-implemented method as claimed in claim 1, wherein determining entity mappings further comprises determining interacting entity pairs from the first set of entities, the second set of entities, entity family of the graph structure, and/or disease selection information, wherein an entity pair comprises at least a first entity and a second entity and an entity relationship associated therewith.

16. The computer-implemented method as claimed in claim 15, wherein each interacting entity pair comprises one or more from the group of:
an entity of a first type and an entity of a second type having a predicted relationship therewith, wherein the entity of the second type corresponds to an entity type associated with the family entity of the graph structure;
an entity of a first type and an entity of a second type having a known relationship therewith, wherein the entity of the second type corresponds to an entity type associated with the family entity of the graph structure.

17. The computer-implemented method as claimed in claim 11, wherein the step of retrieving the second set of entities further comprising retrieving a set of known interacting entity pairs from the content source.

18. The computer-implemented method as claimed in claim 13, wherein:
the first type of entities correspond to entities associated with drugs, compounds, chem(o)informatics, genes of other drug targets, and/or other therapies;
the second type of entities correspond to entities associated with protein and/or gene families; and
the candidate entities of interest correspond to entities of the first type of entities.

19. The computer-implemented method as claimed in claim 18, wherein the relationship between each entity pair of the set of predicted interacting biological entities comprises a relationship based on the first entity of the first entity type and the second entity of the second entity type in relation to a disease associated with the disease selection information.

20. (canceled)

21. The computer-implemented method as claimed in claim 1, wherein the one or more machine learning (ML) model(s) comprises a relational ML model configured for predicting pairs of interacting entities associated with the disease selection information, wherein the relational ML model is trained based on a machine learning technique using labelled training datasets and/or rulesets for predicting pairs of interacting entities associated with disease selection information,
wherein the one or more ML model(s) are configured for predicting pairs of interacting entity pairs associated with disease selection information from a corpus of text, the corpus of text comprising a large scale document repository including a plurality of documents, articles, literature, web-sites and/or any other digital information and/or data associated with disease selection information, entities of the first type, entities of the second type and/or entities of the second set of entities.

22. (canceled)

23. The computer-implemented method as claimed in claim 1, further comprising detecting clusters of candidate entities of interest based on performing automatic cluster recognition in relation to entities of the first set of entities and the entities of the second set of entities added to the graph structure; and
displaying the clusters of candidate entities of interest as an overlay over the graph structure.

24. (canceled)

25. (canceled)

26. (canceled)

27. A candidate entity identification apparatus comprising a processor unit, a memory unit and a communication interface, the processor unit connected to the memory unit and the communication unit, wherein the apparatus is configured to implement the computer-implemented method according to claim 1.

28. A system comprising:
a user interface configured for receiving data representative of disease selection information;
a candidate entity identification apparatus according to claim 27 connected to the user interface for receiving the disease selection information; and
a display interface configured for displaying the linked graph structure and indications of identified candidate entities of interest.

29. (canceled)
